(19) United States
(12) Patent Application Publication
(10) Pub. No.: US 2009/0186361 A1
(43) Pub. Date: Jul. 23, 2009

(54) METHODS OF PREDICTING THE PHARMACEUTICAL TOXICITY OF TAXANES

(76) Inventors: Andre Rogatko, Atlanta, GA (US); Mark Bouzyk, Lilburn, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/357,672

(22) Filed: Jan. 22, 2009

Related U.S. Application Data
(60) Provisional application No. 61/022,896, filed on Jan. 23, 2008.

(51) Int. Cl. C12Q 1/68

(52) U.S. Cl. 435/6

(57) ABSTRACT

The present disclosure relates to methods of identifying a recipient subject who has an elevated risk of an adverse or toxic reaction to a taxane. The predictive methods of the present disclosure allow for the recipient subject most at risk of adversely reacting to the taxane, to be administered alternative treatments or to have the dosage modified to reduce or eliminate toxic reactions. It has been discovered that recipient subjects bearing certain SNPs of the cytochrome CYP2C8 and TUBB genes have an elevated risk of toxicity from a taxane. The methods of the present disclosure encompass methods of calculating a Toxicity Index for a recipient subject based on clinical data from the recipient subject as it relates to the administration and reaction to treatment with taxane. This Toxicity Index value is then correlated to the presence of polymorphisms at a site in each of the gene loci CYP2C8 and TUBB, or a combination of both sites.

### CROSS-REFERENCE TO RELATED APPLICATIONS

This application claims priority to U.S. Provisional Patent Application Ser. No. 61/022,896, entitled “Predictive Methods for Pharmaceutical Toxicity of Taxanes” filed on Jan. 23, 2008, the entirety of which is hereby incorporated by reference.

### FIELD OF THE INVENTION

The present disclosure relates to single nucleotide polymorphisms in the CYC2C8 and TUBB genes, and to the association of these SNPs alone or in combination, or in combination with SNPs of other genes, that predict the level of toxicity of a taxane.

### BACKGROUND

The effectiveness of drug therapy can be limited by variations in drug toxicity and efficacy mediated, in part, by genetic determinants. Often, individual variation may result from genetic polymorphisms affecting metabolic enzymes or transporters that alter the pharmacokinetic fate of a drug, or in proteins representing drug targets that alter the pharmacodynamics of the drug. Any given compound may be subject to several competing and/or serial metabolic, transport or signaling reactions before being excreted from the body. Thus, functional variation in the proteins that govern these processes has the capacity of profoundly altering drug efficacy and toxicity. Although genetic polymorphisms within drug-metabolizing enzymes have been associated with toxic or inefficacious responses to specific drugs, however, and despite recent advances in pharmacogenetic research, a gap still remains between knowledge about genetic polymorphisms and their clinical significance.

Paclitaxel is widely used in the treatment of breast, lung, prostate, ovarian and aerodigestive cancers. Despite widespread use in the clinic, and extensive knowledge of taxane pharmacokinetics, there are few predictors of toxicity following treatment with paclitaxel. Population pharmacokinetic models have identified recipient subject characteristics (e.g., serum alpha-1 acid glycoprotein levels) to predict docetaxel clearance. However, the link between the characteristics and toxicity has not been established, and the relative contribution of pharmacokinetics (drug exposure) alone as a determinant of toxicity is unknown.

The taxanes are metabolized by the polymorphic enzymes CYP2C8, CYP3A4, and possibly CYP3A5. Serum bilirubin levels may also predict a toxic response to docetaxel even though treatment eligibility requires subjects to have normal or near normal hepatic and renal function (Rogatko et al., (2004) Clin. Cancer Res. 10:4645-4651). Polymorphisms within the human uridinediphosphate glucuronosyltransferase (UGT1A1) gene have been associated with serum bilirubin levels. Therefore, individuals with variant capacity to metabolically inactivate taxanes may be at risk for toxic reactions (low capacity) or inefficacious therapy (high capacity).

Because the determinants of toxic and therapeutic responses to taxanes are unknown, the current treatment paradigm adjusts administered dose(s) based only on body surface area, and does not take into account individual recipient subject genetic and clinical characteristics that may affect pharmacokinetics and pharmacodynamics. If such determinants were known, however, each recipient subject could be maintained at a maximum tolerable dose and duration of therapy, thereby reducing the number of recipient subjects who are underdosed (reduced efficacy) or overdosed (unacceptable toxicity). Significant improvement in treatment outcome would be achieved, especially in the adjuvant setting.

### SUMMARY

The present disclosure relates to methods of identifying a recipient subject who has an elevated risk of an adverse or toxic reaction to a taxane. The predictive methods of the present disclosure allow for the recipient subject most at risk of adversely reacting to the taxane, to be administered alternative treatments or to have the dosage modified to reduce or eliminate toxic reactions.

It has been discovered that recipient subjects bearing certain SNPs of the cytochrome CYP2C8 and TUBB genes have an elevated risk of toxicity from a taxane. The methods of the present disclosure encompass methods of calculating a Toxicity Index for a recipient subject based on clinical data from the recipient subject as it relates to the administration and reaction to treatment with taxane. This Toxicity Index value is then correlated to the presence of polymorphisms at a site in each of the gene loci CYP2C8 and TUBB, or a combination of both sites.

The present disclosure provides methods for identifying the presence or absence of relevant SNPs in a recipient subject, and thereby predicting reactivity of a recipient subject to a taxane before the agent is administered. The methods herein disclosed comprise isolating genomic nucleic acid from a recipient subject and characterizing the isolated DNA as to whether it includes SNPs at polymorphic sites within the CYP2C8 and TUBB gene loci. The homozygosity or heterozygosity of a polymorphic site may then be correlated to a predetermined range of Toxicity Index values associated with taxane administration. An SNP at one or more of the polymorphic sites may indicate that a recipient subject administered a dose of a taxane may have a significantly elevated probability of suffering an adverse reaction to the drug. Consequently, the physician can tailor an administered dose to a lower level, an increased dosage period or select a different version of the taxane to attain a reduced Toxicity Index for that recipient subject. This adjustment may be performed before the recipient subject receives the taxane. The likelihood of a successful treatment combined with less potential harm to the recipient subject is thereby greatly increased.

One aspect of the present disclosure, therefore, provides methods of predicting the toxicity of a taxane to a recipient subject, comprising determining the identity of at least one single nucleotide polymorphism within the genome of the recipient subject, where the identity of the at least one single nucleotide polymorphism has been correlated to a Toxicity Index value associated with the exposure of a recipient subject to a taxane; and then providing a prognostic determination of the level of toxicity of a taxane on the recipient subject. In embodiments of this aspect of the disclosure, a one single nucleotide polymorphism may be a first single nucleotide polymorphism within a gene locus selected from the group consisting of a CYP2C8 gene locus and a TUBB gene locus.

In embodiments of the disclosure, the first single nucleotide polymorphism within a CYP2C8 gene locus has the GenBank SNP Accession No. rs1058932, and wherein if the recipient subject is heterozygous TC at the first single nucleotide polymorphism, the recipient subject has an elevated probability of susceptibility to a taxane toxicity; and the second single nucleotide polymorphism within a TUBB gene locus has the GenBank SNP Accession No. 3132584, and wherein if the recipient subject is heterozygous CA at the first single nucleotide polymorphism, the recipient subject has an elevated probability of susceptibility to a taxane toxicity.

Another aspect of the present disclosure provides for kits that may be used for obtaining data that may predict a level of toxicity of a taxane to a recipient subject by determining whether the recipient subject has at least one single nucleotide polymorphism in a gene locus, where an identity of the single nucleotide polymorphism(s) is correlated to a Toxicity Index of the recipient subject to a taxane, thereby providing a prognostic determination of the toxicity of a taxane to the recipient subject, the kit comprising at least one vessel containing at least one primer oligonucleotide, and instructions for using the primer oligonucleotide to determine the identity of a single nucleotide polymorphism in at least one gene locus of a nucleic acid sample isolated from the recipient subject.

In embodiments of this aspect of the disclosure, the kits may further comprise a plurality of oligonucleotides configured for determining whether the recipient subject has at least one single nucleotide polymorphism in a gene locus or a plurality of gene loci, wherein the gene locus, or the plurality of gene loci, may be selected from the group consisting of TUBB, CYP2C8, MAPT, CYP3A5, or a combination thereof, and correlating the identity of the single nucleotide polymorphism or a plurality of single nucleotide polymorphisms to a Toxicity Index of the recipient subject to a taxane.

The present disclosure, therefore, identifies from among a number of SNPs within the CYP2C8 and TUBB gene loci, certain polymorphisms that correlate to probable Toxicity Index values for administered taxane. The present disclosure, therefore, also provides novel primers and probes that may be useful in characterizing the genotype of a recipient subject as it relates to the CYP2C8 and TUBB SNPs, which may then be correlated to Toxicity Index values.

The present disclosure further provides kits that include one or more of the primers and probes useful for identifying SNPs in the CYP2C8 and TUBB gene loci, and instructions for the use thereof. Embodiments of the kits may also include instructions for correlating the SNP complement of these two genes with predicted Toxicity Index values using the methods described in the present disclosure.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 is a graph illustrating the individual value plots of the Toxicity Index (TI) values calculated for 30 recipient subjects and plotted against their respective CYP2C8 (rs1058932) and TUBB (rs3132584) polymorphisms.

FIG. 2A is a graph illustrating the normal probability plot of the residuals for TI1.

FIG. 2B is a graph illustrating the residuals versus the fitted values for TI1.

FIG. 2C is a graph illustrating a histogram of the residuals for TI1.

FIG. 2D is a graph illustrating the residuals versus the order of the data for TI1.

FIG. 3 illustrates the sequence from human mRNA (GenBank Accession No. NM_000770; Adjei et al., (2009) Antimicrob. Agents Chemother. 52:4400-4406) (SEQ ID NO: 1) encoding the region flanking the CYP2C8 SNP rs1058932 (indicated by the vertical arrow at position 1592 thereof). The forward and reverse PCR primers (SEQ ID NOs: 3 and 4, respectively, and a single-base extension primer sequence (SEQ ID NO: 7) are also indicated.

FIG. 4 illustrates the sequence from human mRNA encoding the region flanking the TUBB SNP (SEQ ID NO: 2). The forward and reverse PCR primers (SEQ ID NOs: 5 and 6, respectively, and a single-base extension primer sequence (SEQ ID NO: 8) are also indicated.

FIGS. 5A and 5B show dot plot graphs of Toxicity Indexes at each of 21 cycles. FIG. 5A: TI1-TI21 for paclitaxel; FIG. 5B: TI1-TI21 for docetaxel. Each symbol represents up to three observations.

FIGS. 6A and 6B show scatter plots of Toxicity Index (TI) versus cycle for each recipient subject. FIG. 6A: paclitaxel; FIG. 6B: docetaxel.

FIGS. 7A and 7B show box plot graphs of TI1-TI5 for each treatment. FIG. 7A: Treatment with paclitaxel; FIG. 7B: treatment with docetaxel. Univariate linear regression analysis was applied to Toxicity Index in the first five cycles since more than 50% recipient subjects showed Toxicity Index in those cycles. Covariates in the analysis included recoded binary demographic variables (sex, race, site, grade, stage) and recoded binary genetic variables.

FIG. 8A shows histograms of the frequency of a Toxicity Index at each of treatment cyclesTI1, TI2, TI3, TI4, and TI5 for paclitaxel.

FIG. 8B shows histograms of the frequency of a Toxicity Index at each of treatment cyclesTI1, TI2, TI3, TI4, and TI5 for docetaxel.

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein cited references”), as well as each document or reference cited in each of the herein-cited references (including any manufacturer's specifications, instructions, etc.), is hereby expressly incorporated herein by reference. Publication dates may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

The term “complementarity” or “complementary” as used herein refers to a sufficient number in the oligonucleotide of complementary base pairs in its sequence to interact specifically (hybridize) with the target nucleic acid sequence of the gene polymorphism to be amplified or detected. As known to those skilled in the art, a very high degree of complementarity is needed for specificity and sensitivity involving hybridization, although it need not be 100%. Thus, for example, an oligonucleotide that is identical in nucleotide sequence to an oligonucleotide disclosed herein, except for one base change or substitution, may function equivalently to the disclosed oligonucleotides. A “complementary DNA” or “cDNA” gene includes recombinant genes synthesized by reverse transcription of messenger RNA (“mRNA”).

The term “cyclic polymerase-mediated reaction” as used herein refers to a biochemical reaction in which a template molecule or a population of template molecules is periodically and repeatedly copied to create a complementary template molecule or complementary template molecules, thereby increasing the number of the template molecules over time.

The term “cycle” as used herein refers to a period of administering a therapeutic agent, particularly a taxane, more particularly paclitaxel or docetaxel, to a human subject in need thereof. The cycle may be for any period of treatment, even extending over a period of three weeks or more. Recipient subjects receiving the dose cycle may receive more than one cycle, most typically between one and six cycles of treatment, less typically twenty cycles or more, depending on the severity of the cancer and the frequency and severity of recurrencies.

The term “denaturation” of a template molecule as used herein refers to the unfolding or other alteration of the structure of a template so as to make the template accessible to duplication. In the case of DNA, “denaturation” refers to the separation of the two complementary strands of the double helix, thereby creating two complementary, single stranded template molecules. “Denaturation” can be accomplished in any of a variety of ways, including by heat or by treatment of the DNA with a base or other denaturant.

The term “detectable amount of product” as used herein refers to an amount of amplified nucleic acid that can be detected using standard laboratory tools. A “detectable marker” refers to a nucleotide analog that allows detection using visual or other means. For example, fluorescently labeled nucleotides can be incorporated into a nucleic acid during one or more steps of a cyclic polymerase-mediated reaction, thereby allowing the detection of the product of the reaction using, e.g., fluorescence microscopy or other fluorescence-detection instrumentation.

The term “detectable moiety” as used herein refers to a label molecule (isotopic or non-isotopic) which is incorporated indirectly or directly into an oligonucleotide, wherein the label molecule facilitates the detection of the oligonucleotide in which it is incorporated, for example when the oligonucleotide is hybridized to amplified gene polymorphisms sequences. Thus, “detectable moiety” is used synonymously with “label molecule”. Synthesis of oligonucleotides can be accomplished by any one of several methods known to those skilled in the art. Label molecules, known to those skilled in the art as being useful for detection, include chemiluminescent or fluorescent molecules. Various fluorescent molecules are known in the art which are suitable for use to label a nucleic acid for the method of the present disclosure. The protocol for such incorporation may vary depending upon the fluorescent molecule used. Such protocols are known in the art for the respective fluorescent molecule.

The term “detectably labeled” as used herein refers to a fragment or an oligonucleotide that contains a nucleotide that is radioactive, or that is substituted with a fluorophore, or that is substituted with some other molecular species that elicits a physical or chemical response that can be observed or detected by the naked eye or by means of instrumentation such as, without limitation, scintillation counters, calorimeters, UV spectrophotometers and the like. As used herein, a “label” or “tag” refers to a molecule that, when appended by, for example, without limitation, covalent bonding or hybridization, to another molecule, for example, also without limitation, a polynucleotide or polynucleotide fragment, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence, electrochemiluminescence, raman, calorimetric, hybridization protection assay, and mass spectrometry

The term “DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

The term “DNA” as used herein refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

The terms “enzymatically amplify” or “amplify” as used herein refers to DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme Qβ replicase and a ribonucleic acid (RNA) sequence template attached to a probe complementary to the DNA to be copied which is used to make a DNA template for exponential production of complementary RNA; strand displacement amplification (SDA); Qβ replicase amplification (QPRA); self-sustained replication (3SR); and NASBA (nucleic acid sequence-based amplification), which can be performed on RNA or DNA as the nucleic acid sequence to be amplified.

The term “fragment” of a molecule such as a protein or nucleic acid as used herein refers to any portion of the amino acid or nucleotide genetic sequence.

The term “genome” as used herein refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule).

The term “heterozygous” or “heterozygous polymorphism” as used herein refers to when two alleles of a diploid cell or organism at a given locus are different, that is, that they have a different nucleotide exchanged for the same nucleotide at the same place in their sequences.

The term “homozygous” or “homozygous polymorphism” is meant that the two alleles of a diploid cell or organism at a given locus are identical, that is, that they have the same nucleotide for nucleotide exchange at the same place in their sequences.

The term “hybridization” or “hybridizing,” as used herein refers to the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequenced has a T, G, C or A, respectively. The hybridized fragment/oligonucleotide is called a “duplex.”

The term “hybridization complex” as used herein refers to a sandwich assay, means a complex of nucleic acid molecules including at least the target nucleic acid and a sensor probe. It may also include an anchor probe.

The term “hybridizing under stringent conditions” as used herein refers to annealing a first nucleic acid to a second nucleic acid under stringent conditions as defined below. Stringent hybridization conditions typically permit the hybridization of nucleic acid molecules having at least 70% nucleic acid sequence identity with the nucleic acid molecule being used as a probe in the hybridization reaction. For example, the first nucleic acid may be a test sample or probe, and the second nucleic acid may be the sense or antisense strand of an ovomucoid gene expression control region or a fragment thereof. Hybridization of the first and second nucleic acids may be conducted under stringent conditions, e.g., high temperature and/or low salt content that tend to disfavor hybridization of dissimilar nucleotide sequences. Alternatively, hybridization of the first and second nucleic acid may be conducted under reduced stringency conditions, e.g. low temperature and/or high salt content that tend to favor hybridization of dissimilar nucleotide sequences. Low stringency hybridization conditions may be followed by high stringency conditions or intermediate medium stringency conditions to increase the selectivity of the binding of the first and second nucleic acids. The hybridization conditions may further include reagents such as, but not limited to, dimethyl sulfoxide (DMSO) or formamide to disfavor still further the hybridization of dissimilar nucleotide sequences. A suitable hybridization protocol may, for example, involve hybridization in 6×SSC (wherein 1×SSC comprises 0.015 M sodium citrate and 0.15 M sodium chloride), at 65° C. in an aqueous solution, followed by washing with 1×SSC at 65° C. Formulae to calculate appropriate hybridization and wash conditions to achieve hybridization permitting 30% or less mismatch between two nucleic acid molecules are disclosed, for example, in Meinkoth et al., (1984) Anal. Biochem. 138:267-284; the contents of which is incorporated herein by reference in its entirety. Protocols for hybridization techniques are well known to those of skill in the art and standard molecular biology manuals may be consulted to select a suitable hybridization protocol without undue experimentation. See, for example, Sambrook et al., 1989, “Molecular Cloning: A Laboratory Manual,” 2nd ed., Cold Spring Harbor Press: the contents of which is incorporated herein by reference in its entirety.

Typically, stringent conditions will be those in which the salt concentration is less than about 1.5 M Na ion, typically about 0.01 to 1.0 M Na ion concentration (or other salts) from about pH 7.0 to about pH 8.3 and the temperature is at least about 30° C for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC at 50 to 55° C. Exemplary moderate stringency conditions include hybridization in 40 to 45% formamide, 1 M NaCl, 1% SDS at 37° C., and a wash in 0.5 x to 1×SSC at 55 to 60° C. Exemplary high stringency conditions include hybridization in 50% formamide, 1 M NaCl, 1% SDS at 37° C., and a wash in 0.1×SSC at 60 to 65° C.

The term “immobilized on a solid support” as used herein refers to a fragment, primer or oligonucleotide is attached to a substance at a particular location in such a manner that the system containing the immobilized fragment, primer or oligonucleotide may be subjected to washing or other physical or chemical manipulation without being dislodged from that location. A number of solid supports and means of immobilizing nucleotide-containing molecules to them are known in the art; any of these supports and means may be used in the methods of this disclosure.

The term “locus” or “loci” as used herein refers to the site of a gene on a chromosome. A single allele from each locus is inherited from each parent. Each recipient subject's particular combination of alleles is referred to as its “genotype”. Where both alleles are identical, the individual is homozygous for the trait controlled by that pair of alleles; where the alleles are different, the individual is the to be heterozygous for the trait.

The term “melting temperature” as used herein refers to a temperature at which hybridized duplexes dehybridize and return to their single-stranded state. Likewise, hybridization will not occur in the first place between two oligonucleotides, or, herein, an oligonucleotide and a fragment, at temperatures above the melting temperature of the resulting duplex. It is presently advantageous that the difference in melting point temperatures of oligonucleotide-fragment duplexes of this disclosure be from about 1° C. to about 10° C. so as to be readily detectable.

The term “nucleic acid molecule” as used herein is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

The term “oligonucleotide” as used herein refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

The term “polymerase chain reaction” or “PCR” as used herein refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

The term “polymerase” as used herein refers to an enzyme that catalyzes the sequential addition of monomeric units to a polymeric chain, or links two or more monomeric units to initiate a polymeric chain. In advantageous embodiments of this disclosure, the “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

The term “polynucleotide” as used herein refers to a linear chain of nucleotides connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides liked by a phosphodiester backbone. A “modified polynucleotide” refers to a polynucleotide in which one or more natural nucleotides have been partially or substantially replaced with modified nucleotides.

The term “primer” as used herein refers to an oligonucleotide, at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template, i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

The term “probes” as used herein refers to oligonucleotides nucleic acid sequences of variable length, used in the detection of identical, similar, or complementary nucleic acid sequences by hybridization. An oligonucleotide sequence used as a detection probe may be labeled with a detectable moiety. Various labeling moieties are known in the art. The moiety may, for example, either be a radioactive compound, a detectable enzyme (e.g. horse radish peroxidase (HRP)) or any other moiety capable of generating a detectable signal such as a calorimetric, fluorescent, chemiluminescent or electrochemiluminescent signal. The detectable moiety may be detected using known methods.

The term “protein” as used herein refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

The term “restriction enzyme” as used herein refers to an endonuclease (an enzyme that cleaves phosphodiester bonds within a polynucleotide chain) that cleaves DNA in response to a recognition site on the DNA. The recognition site (restriction site) may be, but is not limited to, a specific sequence of nucleotides typically about 4-8 nucleotides long.

The term “single nucleotide polymorphism” or “SNP” as used herein refers to polynucleotide that differs from another polynucleotide by a single nucleotide exchange. For example, without limitation, exchanging one A for one C, G, or T in the entire sequence of polynucleotide constitutes a SNP. Of course, it is possible to have more than one SNP in a particular polynucleotide. For example, at one locus in a polynucleotide, a C may be exchanged for a T, at another locus a G may be exchanged for an A, and so on. When referring to SNPs, the polynucleotide is most often DNA.

The term “taxane” as used herein refers to diterpenes produced by plants of the genus Taxus (yews) or synthetic versions or derivatives thereof. As their name suggests, they were first derived from natural sources, but some have been synthesized artificially. Taxanes include, but are not limited to, paclitaxel (TAXOL™) ((2aR,4S,4aS,6R,9S,11S,12S,12aR,12bS)-1,2a,3,4,4a,6,9,10,11,12,12a,12b-Dodecahydro-4,6,9,11,12,12b-hexahydroxy-4a,8,13,13-tetramethyl-7,11-methano-5H-cyclodeca(3,4)benz(1,2-b)oxet-5-one 6,12b-diacetate, 12-benzoate, 9-ester with (2R,3S)-N-benzoyl-3-phenylisoserine, and docetaxel (TAXOTERE™) (Benzenepropanoic acid, β-(((1,1-dimethylethoxy)carbonyl)amino)-α-hydroxy-, 12b-(acetyloxy)-12-(benzoyloxy)-2a,3,4,4a,5,6,9,10,11,12,12a,12b-dodecahydro-4,6,11-trihydroxy-4a,8,13,13-tetramethyl-5-oxo-7,11-methano-1H-cyclodeca(3,4)benz(1,2-b)oxet-9-yl ester trihydrate, (2aR-(2aα,4β,4aβ,6β,9(αR*,βS),11α,12α,12aα,12bα))-,. Paclitaxel was originally derived from the Pacific yew tree.

The term “template” as used herein refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such DNA strand may be single-stranded or it may be part of a double-stranded DNA template. In applications of the present disclosure requiring repeated cycles of polymerization, e.g., the polymerase chain reaction (PCR), the template strand itself may become modified by incorporation of modified nucleotides, yet still serve as a template for a polymerase to synthesize additional polynucleotides.

The term “thermocyclic reaction” as used herein refers to a multi-step reaction wherein at least two steps are accomplished by changing the temperature of the reaction.

The term “thermostable polymerase” as used herein refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include Taq, Tth, Pfu, Vent, deep vent, UlTma, and variations and derivatives thereof.

The term “variance” as used herein refers to a difference in the nucleotide sequence among related polynucleotides. The difference may be the deletion of one or more nucleotides from the sequence of one polynucleotide compared to the sequence of a related polynucleotide, the addition of one or more nucleotides or the substitution of one nucleotide for another. The terms “mutation,” “polymorphism” and “variance” are used interchangeably herein. As used herein, the term “variance” in the singular is to be construed to include multiple variances; i.e., two or more nucleotide additions, deletions and/or substitutions in the same polynucleotide. A “point mutation” refers to a single substitution of one nucleotide for another.

Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

### Description

The present disclosure encompasses methods for the predicting the likelihood of a toxic reaction in a recipient subject administered a taxane. The present disclosure provides methods of predicting the likelihood of an adverse reaction developing in a recipient subject when administered a taxane, the methods being based on the presence or absence of certain SNPs in the CYC2C8 and TUBB gene loci. CYC2C8 is an isozyme of cytochrome P450 and mediates hepatic metabolism of taxanes such as, but not limited to, paclitaxel, docetaxel and the like. TUBB encodes a tubulin of the microtubulin system of the cell. The methods of the present disclosure can be used to determine whether an individual recipient subject possesses, within these two genetic loci the SNPs as described herein and to predict therefrom the potential toxicity of the taxane to the recipient subject before delivery to the recipient subject. With this knowledge, a physician can modify the treatment regimen to deliver a therapeutically effective taxane dose while reducing the toxic effects of the drug experienced by the recipient subject to more tolerable levels.

In one aspect, therefore, the present disclosure relates to the identification of single nucleotide polymorphisms (SNPs) in the genome of a recipient subject, and to methods for the identification of a recipient subject carrying specific alleles of these SNPs that can be associated with taxane toxicity. In a further aspect, the present disclosure relates to the association of SNPs in the CYC2C8 and TUBB loci with the toxic effect of a taxane on an individual recipient subject. The present disclosure also provides oligonucleotides that can be used as primers to amplify those specific nucleic acid sequences of the CYC2C8 and TUBB genes encompassing the SNP's associated with a calculated Toxicity Index (TI) value, and oligonucleotides that can be used as probes in the detection of relevant nucleic acid sequences and the SNPs thereof within the CYC2C8 and TUBB genes.

It has been discovered that two SNPs, rs1058932 within the CYC2C8 gene, and rs3132584 within the TUBB locus, are associated with the degree of toxicity to a taxane experienced by the recipient subject to whom it is administered. The SNP termed rs1058932 of the CYC2C8 gene constitutes a cytosine (C)—thymine (T) substitution (C/T) within the CYC2C8 gene. The SNP termed, rs3132584, within the TUBB gene constitutes a cytosine (C) to adenosine (A) substitution (C/A substitution) of the TUBB gene.

It has further been discovered that recipient subjects bearing certain SNPs of the cytochrome P450 CYP2C8 and TUBB2 genes have an elevated risk of toxicity from a taxane as indicated by a Toxicity Index value. Methods according to this disclosure first involve the derivation of a Toxicity Index for a particular taxane based on clinical data from recipient subjects as it relates to the administration and subsequent reaction to treatment with taxane (se, for example, Example 1, below). This Toxicity Index value may then be correlated to the presence of polymorphisms at certain sites in each of the CYP2C8 and the TUBB loci, a combination of both sites. It is contemplated that it is within the scope of the present disclosure for multiple SNPs within these loci to be associated with the TI value for a taxane. Accordingly, the methods of the present disclosure allow for the determination of the genotype of a recipient subject, at least with respect to SNPs within the CYP2C8 and TUBB loci, before administering a taxane to the recipient subject in need, thereby providing a painless and relatively simple means of determining the potential toxicity of the taxane to a recipient subject. The treatment dosage protocol may then be adjusted to the specific needs of the recipient subject to minimize the toxic effects of the therapeutic agent while preserving its pharmaceutical efficacy.

Methods for calculating the Toxicity Index of a recipient subject, or group of recipient subjects, with respect to a specific taxane are described in Example 1 below. The present disclosure further provides methods for the identification in a subject recipient subject of the presence or absence of the relevant SNPs correlating to the TI of a taxane, and providing a means of predicting the reactivity of a recipient subject to a taxane before the agent is administered. The methods herein disclosed may, therefore, comprise the steps of isolating from a recipient subject or recipient subjects genomic nucleic acid and characterizing the isolated nucleic acid as to whether it includes SNPs at one or both of the polymorphic sites within the CYP2C8 and TUBB loci. The homozygosity and/or heterozygosity of the polymorphic sites may then be correlated to the predetermined range of Toxicity Index values associated with a taxane. Having an SNP at one or both of the polymorphic sites may indicate that a recipient subject, if administered a dose of a taxane at a level typically administered, may have a significantly elevated probability of suffering an adverse reaction to the drug. Consequently, the physician can provide the taxane at a lower dosage over an increased treatment period, or select a different version of the taxane more acceptable to the recipient subject. This may be performed before the recipient subject receives the taxane. The likelihood of a successful treatment combined with less potential harm to the recipient subject is thereby greatly increased. It is contemplated that, while being demonstrated using paclitaxel or docetaxel, the methods of the present disclosure are applicable to any taxol derivative that may be therapeutically effective against a cancer in a subject recipient subject, but also may have undesirable toxic effects.

The present disclosure, therefore, identifies from among a number of previously identified SNPs within the CYP2C8 and TUBB genes, certain polymorphisms that have a surprising correlation to probable Toxicity Index values for administered taxanes. The present disclosure, additionally provides novel primers and probes that are useful in characterizing the genotype of a recipient subject as it relates to the CYP2C8 and TUBB SNPs, and which may then be correlated to Toxicity Index values.

The present disclosure further provides kits that may include, but are not limited to, one or more of the primers and probes for identifying specific SNPs in the CYP2C8 and TUBB genes, and instructions for the uses thereof. Embodiments of the kits may also include instructions for correlating the SNP complement of these two genes with predicted Toxicity Index values using the methods described in the present disclosure.

### Tissue and DNA Samples

To determine the genotype of a recipient subject according to the methods of the present disclosure, it is necessary to obtain a sample of genomic DNA from that recipient subject. Typically, that sample of genomic DNA will be obtained from a sample of tissue or cells taken from that recipient subject.

The tissue sample can comprise hair (including roots), buccal (cheek) swabs, blood, saliva, semen, embryos, muscle or any internal organs. In the methods of the present disclosure, the source of the tissue sample, and thus also the source of the test nucleic acid sample, is not critical. For example, the test nucleic acid can be obtained from cells within a body fluid, or from cells constituting a body tissue. The particular body fluid from which cells are obtained is also not critical to the present disclosure. For example, the body fluid may be selected from the group consisting of blood, ascites, pleural fluid and spinal fluid. Furthermore, the particular body tissue from which cells are obtained is also not critical to the methods of the present disclosure. For example, the body tissue may be selected from the group consisting of skin, endometrial, uterine and cervical tissue. Both normal and tumor tissues can be used. A useful source of genomic DNA can be obtained by a scraping of cells from the interior surface of the oral cavity. A particularly useful source of DNA is whole-blood withdrawn from the recipient subject by procedures well-known to those in the art. These non-invasive procedures are rapid and painless with no discomfort to the recipient subject. This procedure readily allows samples to be obtained from a plurality of recipient subjects.

Typically, the tissue sample may be marked with an identifying number or other indicia that relates the sample to the individual recipient subject from which the sample was taken. The identity of the sample advantageously remains constant throughout the methods of the disclosure thus guaranteeing the integrity and continuity of the sample during extraction and analysis. Alternatively, the indicia may be changed in a regular fashion that ensures that the data, and any other associated data, can be related back to the recipient subject from whom the data was obtained.

The amount/size of the sample required is known to those skilled in the art. For example, non-limiting examples of sample sizes/methods include hair roots: greater than five and less than twenty; buccal swabs: 15 to 20 seconds of rubbing with modest pressure in the area between outer lip and gum using one Cytosoft® cytology brush; bone: 0.0020 g to 0.0040 g; and blood: 30 to 70 μl, although more typical withdrawn samples may be from about 1 ml to about 10 mls in volume.

Generally, the tissue sample is placed in a container that is labeled using a numbering system bearing a code corresponding to the recipient subject, for example. Accordingly, the genotype of a particular recipient subject is easily traceable at all times.

DNA can be isolated from the tissue/cells by techniques known to those skilled in the art (see, e.g., U.S. Pat. Nos. 6,548,256 and 5,989,431, Hirota et al., (1989) Jinrui Idengaku Zasshi. 34:217-223 and John et al., (1991) Nuc. Acids Res. 19:408; the disclosures of which are incorporated herein by reference in their entireties). For example, high molecular weight DNA may be purified from cells or tissue using proteinase K extraction and ethanol precipitation. DNA may be extracted from a recipient subject's specimen using any other suitable methods known in the art.

### Determining the Genotype of a Recipient Subject

The present disclosure provides methods to determine the genotype of a given recipient subject of interest, in order to identify if that recipient subject is carrying specific alleles of the SNPs of the disclosure that are predictive of taxane toxicity. There are many methods known in the art for determining the genotype of a recipient subject and for identifying whether the given DNA sample contains a particular SNP. Any method for determining genotype can be used in the present disclosure.

Detection or determination of a nucleotide identity or the genotype of one or more single nucleotide polymorphism(s) (SNP typing), may be accomplished by any one of a number methods or assays known in the art. Many DNA typing methodologies are useful for allelic discrimination and detection of SNPs. Furthermore, the products of allelic discrimination reactions or assays may be detected by one or more detection methods. The majority of SNP genotyping reactions or assays can be assigned to one of four broad groups (allele-specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, allele-specific hybridization involves a hybridization probe that is capable of distinguishing between two DNA targets differing at one nucleotide position by hybridization. Probes may be designed with the polymorphic base in a central position in the probe sequence, whereby under optimized hybridization assay conditions only the perfectly matched probe target hybrids are stable and hybrids with a one base mismatch are unstable. Another strategy that couples detection and allelic discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Primer extension reactions (i.e., mini-sequencing, allele-specific extensions, or simple PCR amplification) are particularly useful in the allelic discrimination reactions of the methods of the present disclosure. For example, in mini sequencing a primer anneals to its target DNA region immediately upstream of the SNP and is extended with a single nucleotide complementary to one allele of the polymorphic site. There is, however, no extension in the absence of nucleotide complementary. The single base incorporated can be detected by labeling the incoming nucleotide beforehand. Incorporation of the label onto the primer, therefore, indicates which of the alleles is present in the target DNA.

Oligonucleotide ligation assays require two allele-specific probes and one common ligation probe per SNP. The common ligation probe hybridizes adjacent to an allele-specific probe and when there is a perfect match of the appropriate allele-specific probe a ligase joins both allele-specific and the common probes. In the absence of a perfect match, the ligase is unable to join the allelic specific and common probes.

Alternatively, an invasive cleavage method requires an oligonucleotide called an invader probe and allele-specific probes to anneal to the target DNA with an overlap of one nucleotide. When the allele-specific probe is complementary to the polymorphic base, overlaps of the 3′ end of the invader oligonucleotide form a structure that is recognized and cleaved by a Flap endonuclease releasing the 5′ arm of the allele-specific probe.

5′-exonuclease activity of the TAQMAN™ assay (Applied Biosystems, Inc) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the major allele and the other to the minor allele. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragment. Mismatched probes are displaced without fragmentation. The genotype of a sample is determined by measuring the signal intensity of the two different dyes.

It will be appreciated that numerous other methods for allelic discrimination and detection are known in the art and some of which are described in further detail below. It will also be appreciated that reactions such as arrayed primer extension mini sequencing, tag microarrays and allelic specific extension could be performed on a microarray. One such array based genotyping platform is the microsphere based tag-it high throughput genotyping array (Bortolin et al., (2004) Clinical Chemistry 50:2028-2036). This method amplifies genomic DNA by PCR followed by allele-specific primer extension with universally tagged genotyping primers. The products are then sorted on a Tag-It array and detected using the Luminex xMAP system.

More specifically, SNP-typing methods that may be used in the genotyping methods of the disclosure include, but are not limited to, the following:

(a) Restriction Fragment Length Polymorphism (RFLP) strategy: Briefly, a short segment of DNA (typically several hundred base pairs) is amplified by PCR. Where possible, a specific restriction endonuclease is chosen that cuts the short DNA segment when one variant allele is present but does not cut the short DNA segment when the other allele variant is present. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the initial DNA sample had the allele, which could be cut by the chosen restriction endonuclease. In contrast, if only one higher molecular weight band is observed (at the molecular weight of the PCR product) then the initial DNA sample had the allele variant that could not be cut by the chosen restriction endonuclease. Finally, if both the higher molecular weight band and the two lower molecular weight bands are visible then the initial DNA sample contained both alleles, and therefore the subject was heterozygous for this single nucleotide polymorphism

(b) Sequencing: for example the Maxam-Gilbert technique for sequencing (Maxam & Gilbert, (1977) Proc. Natl. Acad. Sci. USA 74:560-564) or the dideoxy method of sequencing (Sanger et al., (1977) Proc. Natl. Acad. Sci. USA 74:5463-5467) may be used.

(c) Single nucleotide extension: A most advantageous method for use in the methods of the present disclosure is single base extension. Prior knowledge of the location of the targeted SNP within the parent sequence is necessary. Single Base Extension (SBE) otherwise known as mini-sequencing, requires the annealing of a primer one base upstream of the base to be determined, i.e., the base that is polymorphic. After annealing of the primer to the region of the target DNA immediately upstream of the polymorphic base, the primer is extended by one base with a labeled ddNTP, thereby ending the extension. The final product may be separated. The label of the incorporated ddNTP may be, for example, but not limited to, a fluorescent or radioactive tag.

In a mini-sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP, or only ddNTP, in the mini sequencing reaction depends on the method chosen for detecting the products. Such methods include, but are not limited to, hybridization methods as described in the U.S. Pat. Nos. 6,270,961 & 6,025,136; template-directed dye-terminator incorporation with fluorescent polarization-detection (TDI-FP) as described by Freeman et al., ((2002) J. Mol. Diagnostics 4:209-215) for large scale screening of SNPs; oligonucleotide ligation assay (OLA) based on ligation of probe and detector oligonucleotides annealed to a polymerase chain reaction amplicon strand with detection by an enzyme immunoassay (Villhermosa (2001) J. Hum. Virol. 4:238-248; Romppanen (2001) Scand. J. Clin. Lab. Invest. 61:123-129; Iannone et al., (2000) Cytometry 39:131-140); Ligation-Rolling Circle Amplification (L-RCA) as described in Qi et al., (2001) Nuc. Acids Res. 29:E116; 5′-nuclease assay as described by Aydin et al., (2001) Biotechniques 4:920-922, 924, 926-928; polymerase proofreading methods as described in WO 0181631; detection of single base pair DNA mutations by enzyme-amplified electronic transduction as described in Patolsky et al., (2001) Nat. Biotech. 19:253-257.

Gene chip technologies are also known for single nucleotide polymorphism discrimination whereby numerous polymorphisms may be tested for simultaneously on a single array (EP 1120646 and Gilles et al. (1999) Nat. Biotechnology 17:365-370); Matrix assisted laser desorption ionization time of flight (MALDI-TOF) mass spectroscopy is also useful in the genotyping single nucleotide polymorphisms through the analysis of micro-sequencing products (Haff & Smirnov (1997) Nuc. Acids Res. 25:3749-3750; Haff & Smirnov (1997) Genome Res. 7:378-388; Sun et al., (2000) Nuc. Acids Res. 28:e68; Braun et al. (1997) Clin. Chem. 43:1151-1158; Little et al., (1997) Eur. J. Clin. Chem. Clin. Biochem. 35:545-548; Fei et al, (2000) Nuc. Acids Res. 26:2827-2828; and Blondal et al., (2003) Nuc. Acids Res. 31:e155). Allele specific PCR methods have also been successfully used for genotyping single nucleotide polymorphisms (Hawkins et al., (2002) Hum. Mutat. 19:543-553).

### Determining the Genotype Using Cyclic Polymerase Mediated Amplification

In certain embodiments of the present disclosure, a step in the detection of a given SNP can be performed using cyclic polymerase-mediated amplification methods. Any one of the methods known in the art for amplification of DNA may be used, such as for example, the polymerase chain reaction (PCR), the ligase chain reaction (LCR) (Barany, F., (1991) Proc. Natl. Acad. Sci. U.S.A. 88:189-193), the strand displacement assay (SDA), or the oligonucleotide ligation assay (“OLA”) (Landegren et al., (1988) Science 241:1077-1080). Nickerson et al. have described a nucleic acid detection assay that combines attributes of PCR and OLA (Nickerson et al., (1990) Proc. Natl. Acad. Sci. U.S.A. 87:8923-8927). Other known nucleic acid amplification procedures, such as transcription-based amplification systems (Malek et al., U.S. Pat. No. 5,130,238; Davey et al., European Patent Application 329,822; Schuster et al., U.S. Pat. No. 5,169,766; Miller et al., PCT Application WO89/06700; Kwoh et al., (1989) Proc. Natl. Acad. Sci. U.S.A. 86:1173; Gingeras et al., PCT Application WO88/10315)), or isothermal amplification methods (Walker et al., (1992) Proc. Natl. Acad. Sci. U.S.A. 89:392-396) may also be used.

The most advantageous method of amplifying DNA fragments containing the SNPs of the disclosure employs PCR (see e.g., U.S. Pat. Nos. 4,965,188; 5,066,584; 5,338,671; 5,348,853; 5,364,790; 5,374,553; 5,403,707; 5,405,774; 5,418,149; 5,451,512; 5,470,724; 5,487,993; 5,523,225; 5,527,510; 5,567,583; 5,567,809; 5,587,287; 5,597,910; 5,602,011; 5,622,820; 5,658,764; 5,674,679; 5,674,738; 5,681,741; 5,702,901; 5,710,381; 5,733,751; 5,741,640; 5,741,676; 5,753,467; 5,756,285; 5,776,686; 5,811,295; 5,817,797; 5,827,657; 5,869,249; 5,935,522; 6,001,645; 6,015,534; 6,015,666; 6,033,854; 6,043,028; 6,077,664; 6,090,553; 6,168,918; 6,174,668; 6,174,670; 6,200,747; 6,225,093; 6,232,079; 6,261,431; 6,287,769; 6,306,593; 6,440,668; 6,468,743; 6,485,909; 6,511,805; 6,544,782; 6,566,067; 6,569,627; 6,613,560; 6,613,560 and 6,632,645; the disclosures of which are incorporated by reference in their entireties), using primer pairs that are capable of hybridizing to the proximal sequences that define or flank a polymorphic site in its double-stranded form.

To perform a cyclic polymerase mediated amplification reaction according to the present disclosure, the primers are hybridized or annealed to opposite strands of the target DNA, the temperature is then raised to permit the thermostable DNA polymerase to extend the primers and thus replicate the specific segment of DNA spanning the region between the two primers. Then the reaction is thermocycled so that at each cycle the amount of DNA representing the sequences between the two primers is doubled, and specific amplification of the gene DNA sequences, if present, results.

Any of a variety of polymerases can be used in the present disclosure. For thermocyclic reactions, the polymerases are thermostable polymerases such as Taq, KlenTaq, Stoffel Fragment, Deep Vent, Tth, Pfu, Vent, and UlTma, each of which are readily available from commercial sources. For non-thermocyclic reactions, and in certain thermocyclic reactions, the polymerase will often be one of many polymerases commonly used in the field, and commercially available, such as DNA pol 1, Klenow fragment, T7 DNA polymerase, and T4 DNA polymerase. Guidance for the use of such polymerases can readily be found in product literature and in general molecular biology guides.

Typically, the annealing of the primers to the target DNA sequence may be carried out for about 2 minutes at about 37-55° C., extension of the primer sequence by the polymerase enzyme (such as Taq polymerase) in the presence of nucleoside triphosphates is carried out for about 3 minutes at about 70-75° C., and the denaturing step to release the extended primer is carried out for about 1 minute at about 90-95° C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65° C., thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g., 200 ng, 500 ng, 1 μg or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In various embodiments, the reaction may comprise 2, 5, 10, 15, 20, 30, 40, 50, or more cycles.

For example, the PCR reaction may be carried out using about 25-50 μl samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl₂, about 10 mM Tris-HCl (pH 9.0), about 50 mM KCl, about 1 μg/ml gelatin, and about 10 μl/ml Triton X-100.

Those of skill in the art are aware of the variety of nucleotides available for use in the cyclic polymerase mediated reactions. Typically, the nucleotides will consist at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

The present disclosure provides oligonucleotides that can be used as primers to amplify specific nucleic acid sequences by polymerase-mediated amplification reactions such as PCR reactions. These primers are especially useful in amplifying nucleic acid regions comprising an SNP in CYP2C8 (such as rs1058932) and in TUBB (such as rs3132584). In certain embodiments, these primers consist of oligonucleotide fragments. Such fragments should be of sufficient length to enable specific annealing or hybridization to the nucleic acid sample. The sequences typically will be about 8 to about 44 nucleotides in length, but may be longer. Longer sequences, e.g., from about 14 to about 50, are advantageous for certain embodiments.

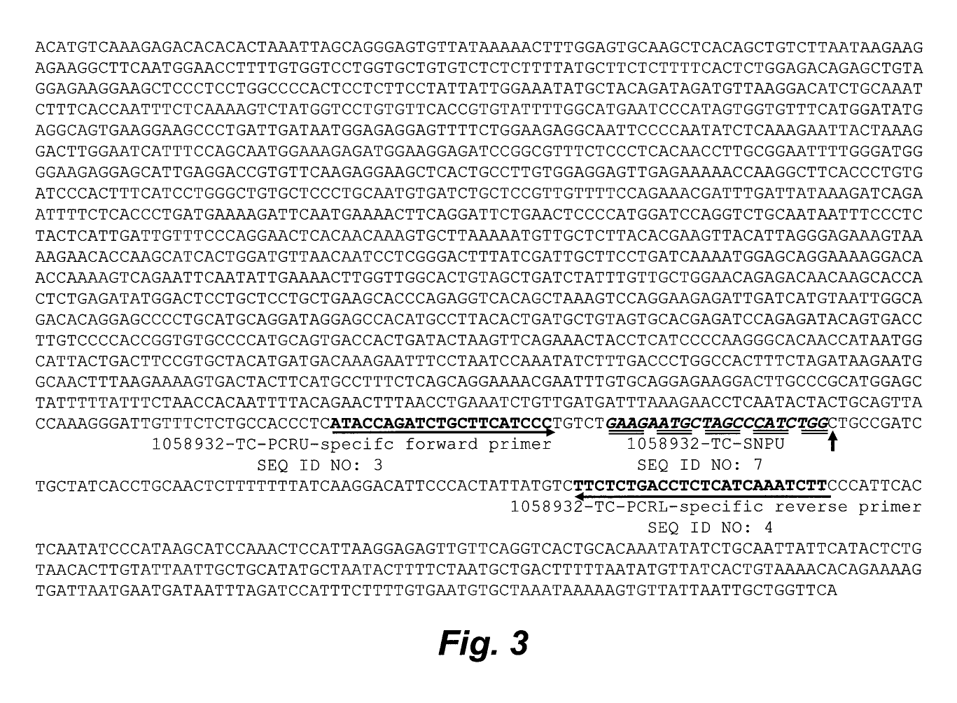

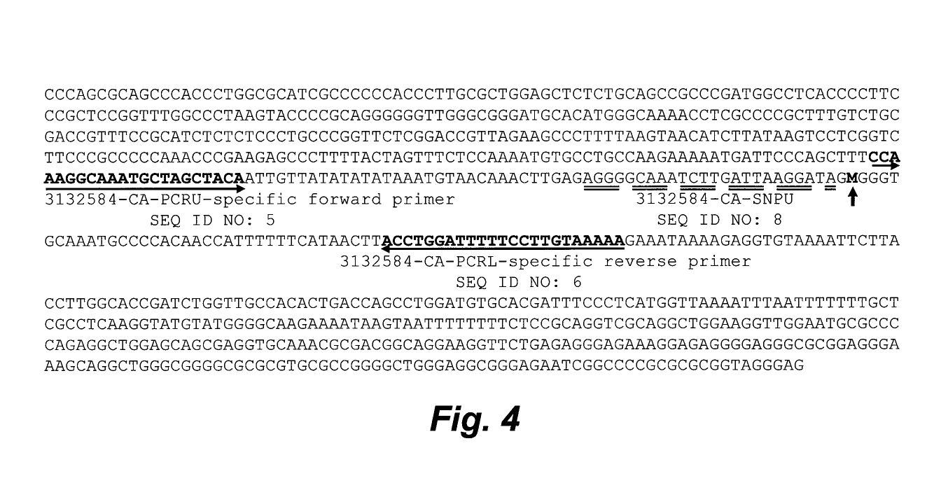

In some embodiments of the disclosure, a fragment of DNA spanning and including the location of the CYP2C8 (rs1058932) polymorphism may be amplified from a nucleic acid template using a forward primer with sequence 5′-ATACCAGATCTGCTTCATCCC-3′ (SEQ ID NO: 3), and a reverse primer with sequence 5′-AAGATTTGATGAGAGGTCAGAGAA-3′ (SEQ ID NO: 4), as indicated in FIG. 3. Similarly, in a preferred embodiment, the nucleic acid region comprising the TUBB) polymorphism rs3132584 may be amplified from a nucleic acid sample using a forward primer having sequence 5′-CAAAGGCAAATGCTAGCTACA-3′ (SEQ ID NO: 5) and a reverse primer having sequence 5′ TTTTTACAAGGAAAAATCCAGGT-3′ (SEQ ID NO: 6), as shown in FIG. 4.

Although various different lengths of primers can be used, and the exact location of the stretch of contiguous nucleotides in the CYP2C8 and TUBB genes used to make the primers can vary, it is important that the sequences to which the forward and reverse primers anneal are located on either side of the particular nucleotide position that is substituted in the SNP to be amplified. For example, when designing primers for amplification of the CYP2C8 (rs1058932) polymorphism, one primer must be located upstream of (not overlapping with) the SNP and the other primer must be located downstream of (not overlapping with) SNP position.

The above methods employ primers located on either side of, and not overlapping with, the SNP in order to amplify a fragment of DNA that includes the nucleotide position at which the SNP is located. Such methods require additional steps, such as sequencing of the fragment, or hybridization of allele-specific probes to the fragment, in order to determine the genotype at the polymorphic site. However, in some embodiments of the present disclosure, the amplification method is itself a method for determining the genotype of the polymorphic site, as for example, in “allele-specific PCR”. In allele-specific PCR, primer pairs are chosen such that amplification itself is dependent upon the input template nucleic acid containing the polymorphism of interest. In such embodiments, primer pairs are chosen such that at least one primer spans the actual nucleotide position of the SNP and is therefore an allele-specific oligonucleotide primer. Typically, the primers may contain a single allele-specific nucleotide at the 3′ terminus preceded by bases that are complementary to the gene of interest. The PCR reaction conditions are adjusted such that amplification by a DNA polymerase proceeds from matched 3′-primer termini, but does not proceed where a mismatch occurs. Allele specific PCR can be performed in the presence of two different allele-specific primers, one specific for each allele, where each primer is labeled with a different dye, for example one allele-specific primer may be labeled with a green dye (e.g., fluorescein) and the other allele-specific primer labeled with a red dye (e.g. sulforhodamine). Following amplification, the products are analyzed for green and red fluorescence. The aim is for one homozygous genotype to yield green fluorescence only, the other homozygous genotype to give red fluorescence only, and the heterozygous genotype to give mixed red and green fluorescence.

Methods for performing allele-specific PCR are well known in the art, and any such methods may be used. For example suitable methods are taught in Myakishev et al., (2001) Genome Research, 1:163-169, Alexander et al., (2004) Mol. Biotechnol. 28:171-174, and Ruano et al., (1989) Nucleic Acids Res. 17:8392, the contents of which are incorporated by reference. In some embodiments of the present disclosure, allele-specific primers may be chosen so that amplification creates a restriction site, facilitating identification of a polymorphic site. To perform, allele-specific PCR the reaction conditions must be carefully adjusted such that the allele-specific primer will only bind to one allele and not the alternative allele, for example, in some embodiments the conditions are adjusted so that the primers will only bind where there is a 100% match between the primer sequence and the DNA, and will not bind if there is a single nucleotide mismatch.

### Compositions and Kits for Detection of the SNPs

The oligonucleotide primers and probes of the present disclosure have commercial applications in diagnostic kits for the detection of the SNPs in specimens. A test kit according to the disclosure may comprise, but is not limited to, any of the oligonucleotide primers or probes according to the disclosure. Such a test kit may additionally comprise one or more reagents for use in cyclic polymerase mediated amplification reactions, such as DNA polymerases, nucleotides (dNTPs), buffers, and the like. An SNP detection kit may also include a lysing buffer for lysing cells contained in the specimen. The kits of the present disclosure may further include instructions for correlating the presence or absence of the targeted SNPs with a Toxicity Index value for a taxane.

A test kit according to this disclosure, for example, may comprise a pair of oligonucleotide primers such as, but not limited to, those having the nucleotide sequences according to SEQ ID NOs: 3-6, and a probe comprising, but not limited to, the nucleotide sequences according to SEQ ID NOs: 7 or 8. In some embodiments such a kit will contain two allele-specific oligonucleotide probes and/or primers useful for single-base extension assays of SNPs such as, but not limited to, SEQ ID NO: 7 and 8. Advantageously, the kit may further comprise additional means, such as reagents, for detecting or measuring the binding or the primers and probes of the present disclosure, and also ideally a positive and negative control.

The present disclosure further encompasses probes according to the present disclosure that are immobilized on a solid or flexible support, such as paper, nylon or other type of membrane, filter, chip, glass slide, microchips, microbeads, or any other such matrix, all of which are within the scope of this disclosure. The probe of this form is now called a “DNA chip”. These DNA chips can be used for analyzing the SNPs of the present disclosure. The present disclosure further encompasses arrays or microarrays of nucleic acid molecules that are based on one or more of the sequences described herein. As used herein “arrays” or “microarrays” refers to an array of distinct polynucleotides or oligonucleotides synthesized on a solid or flexible support, such as paper, nylon or other type of membrane, filter, chip, glass slide, or any other suitable solid support. In one embodiment, the microarray is prepared and used according to the methods and devices described in U.S. Pat. Nos. 5,446,603; 5,545,531; 5,807,522; 5,837,832; 5,874,219; 6,114,122; 6,238,910; 6,365,418; 6,410,229; 6,420,114; 6,432,696; 6,475,808 and 6,489,159 and PCT Publication No. WO 01/45843 A2, the disclosures of which are incorporated by reference in their entireties.

One aspect of the present disclosure, therefore, provides methods of predicting the toxicity of a taxane to a recipient subject, comprising: (a) isolating a nucleic acid sample from a recipient subject; (b) determining a genotype of the recipient subject from the isolated nucleic acid sample, thereby determining the identity of at least one single nucleotide polymorphism within the genome of the recipient subject, wherein the identity of the at least one single nucleotide polymorphism is correlated to a Toxicity Index value associated with the exposure of a recipient subject to a taxane; and (c) providing a prognostic determination of the level of toxicity of a taxane on the recipient subject, where the presence of at least one single nucleotide polymorphism within at least one gene locus correlates to a Toxicity Index value, the Toxicity Index value providing a prognostic determination of the level of toxicity of a taxane on the recipient subject.

In embodiments of this aspect of the disclosure, step (b) of the method may comprise determining the identity of a first single nucleotide polymorphism within a gene locus selected from the group consisting of: a CYP2C8 gene locus and a TUBB gene locus.

In embodiments of this aspect of the disclosure, step (b) of the method may comprise determining the identity of a first single nucleotide polymorphism and a second single nucleotide polymorphism, where the first single nucleotide polymorphism is within a CYP2C8 gene locus, and the second single nucleotide polymorphism is within a TUBB gene locus.

In embodiments of the disclosure, the first single nucleotide polymorphism within a CYP2C8 gene locus has the GenBank SNP Accession No. rs1058932, and wherein if the recipient subject is heterozygous TC at the first single nucleotide polymorphism, the recipient subject has an elevated probability of susceptibility to a taxane toxicity; and the second single nucleotide polymorphism within a TUBB gene locus has the GenBank SNP Accession No. 3132584, and wherein if the recipient subject is heterozygous CA at the first single nucleotide polymorphism, the recipient subject has an elevated probability of susceptibility to a taxane toxicity.

In some embodiments of the methods of this aspect of the disclosure, step (b) may comprise: amplifying a first region of the nucleic acid isolated from the recipient subject using a first forward oligonucleotide primer and a first reverse oligonucleotide primer, wherein the amplified region of the nucleic acid from the recipient subject comprises a first single nucleotide polymorphism site, and wherein the first single nucleotide polymorphism site is associated with a Toxicity Index for a taxane administered to the recipient subject; and determining the nucleotide identity of the first single nucleotide polymorphism site within the first amplified polynucleotide region, thereby determining the Toxicity Index for a taxane administered to the recipient subject.

In other embodiments of the disclosure, step (b) of determining the genotype of a recipient subject may further comprise: amplifying a second region of the nucleic acid isolated from the recipient subject using a second forward oligonucleotide primer and a second reverse oligonucleotide primer, wherein the amplified region of the nucleic acid from the recipient subject comprises a second single nucleotide polymorphism site, and wherein the second single nucleotide polymorphism site is associated with a Toxicity Index for a taxane administered to the recipient subject; and determining the nucleotide identity of the second single nucleotide polymorphism site within the second amplified polynucleotide region, thereby determining the Toxicity Index for a taxane administered to the recipient subject.

In embodiments of the methods of this aspect of the disclosure, the first forward oligonucleotide primer may have a nucleotide sequence selected from the group consisting of SEQ ID NOs: 3, 5, 9, 12, 15, 18, 21, 24, 27, 30, 33, 36, 39, and 42, and the first reverse oligonucleotide primer may have a nucleotide sequence selected from the group consisting of SEQ ID NOs: 4, 6, 10, 13, 16, 19, 22, 25, 28, 31, 34, 37, 40, and 43.

In one embodiment of the disclosure, the first forward oligonucleotide primer may have a nucleotide sequence according to SEQ ID NO: 3 and the first reverse oligonucleotide primer may have a nucleotide sequence according to SEQ ID NO: 4.

In another embodiment of the disclosure, the first forward oligonucleotide primer may have a nucleotide sequence according to SEQ ID NO: 5, and the first reverse oligonucleotide primer may have a nucleotide sequence according to SEQ ID NO: 6.

In the various embodiments of the disclosure, where the step of identifying the nucleotide identity of the first single nucleotide polymorphism site may be by single base extension from an oligonucleotide primer selectively hybridizing to the amplified region under high stringency conditions, the oligonucleotide primer may have a nucleotide sequence selected from the group consisting of SEQ ID NOs: 7, 8, 11, 14, 17, 20, 23, 26, 29, 32, 35, 38, 41, and 44.

In other embodiments of the disclosure, the step of identifying the nucleotide identity of the second single nucleotide polymorphism site may be by single base extension from an oligonucleotide primer selectively hybridizing to the amplified region under high stringency conditions, and the oligonucleotide primer may have a nucleotide sequence selected from the group consisting of SEQ ID NOs: 7, 8, 11, 14, 17, 20, 23, 26, 29, 32, 35, 38, 41, and 44.

In one embodiment, the first single nucleotide polymorphism site is within the CYP28C gene locus, and the nucleotide identity of the single nucleotide polymorphism may be determined by single base extension from an oligonucleotide primer selectively hybridizing to the amplified region under high stringency conditions. In one embodiment of the disclosure, the oligonucleotide primer has a nucleotide sequence according to SEQ ID NO: 7.

In other embodiments of the methods of the disclosure, the second primer oligonucleotide may have a nucleotide sequence according to SEQ ID NO: 5 and the second primer oligonucleotide may have a nucleotide sequence according to SEQ ID NO: 6.

In one embodiment, the first single nucleotide polymorphism site is within the TUBB2 gene locus, and the nucleotide identity of the single nucleotide polymorphism may be determined by single base extension from an oligonucleotide primer selectively hybridizing to the amplified region under high stringency conditions. In these embodiments, the oligonucleotide primer may have a nucleotide sequence according to SEQ ID NO: 8.

In one embodiment of this aspect of the present disclosure, the method may comprise using a first forward oligonucleotide primer having a nucleotide sequence according to SEQ ID NO: 3, a first reverse oligonucleotide primer having a nucleotide sequence according to SEQ ID NO: 4, a second primer oligonucleotide having a nucleotide sequence according to SEQ ID NO: 5, and a second primer oligonucleotide having a nucleotide sequence according to SEQ ID NO: 6, where the first single nucleotide polymorphism site is within the CYP28C gene locus and the second single nucleotide polymorphism site is within the TUBB gene locus, and where the nucleotide identity of the first single nucleotide polymorphism may be determined by single base extension from an oligonucleotide primer having the nucleotide sequence according to SEQ ID NO: 7, and wherein the nucleotide identity of the second single nucleotide polymorphism may be determined by single base extension from an oligonucleotide primer having the nucleotide sequence according to SEQ ID NO: 8.

In the embodiments of this aspect of the disclosure, the Toxicity Index may be determined for a taxane selected from the group consisting of: paclitaxel and docetaxel.

Another aspect of the present disclosure provides for kits that may be used for obtaining data that may predicting a level of toxicity of a taxane to a recipient subject by determining whether the recipient subject has at least one single nucleotide polymorphism in a gene locus, where an identity of the single nucleotide polymorphism(s) is correlated to a Toxicity Index of the recipient subject to a taxane, thereby providing a prognostic determination of the toxicity of a taxane to the recipient subject, the kit comprising at least one vessel containing at least one primer oligonucleotide, and instructions for using the primer oligonucleotide to determine the identity of a single nucleotide polymorphism in at least one gene locus of a nucleic acid sample isolated from the recipient subject.

In embodiments of this aspect of the disclosure, the kits may further comprise a plurality of oligonucleotides configured for determining whether the recipient subject has at least one single nucleotide polymorphism in a gene locus or a plurality of gene loci, wherein the gene locus, or the plurality of gene loci, may be selected from the group consisting of: TUBB, CYP2C8, MAPT, CYP3A5, and a combination thereof, and correlating the identity of the single nucleotide polymorphism or a plurality of single nucleotide polymorphisms to a Toxicity Index of the recipient subject to a taxane.

In some embodiments of this aspect of the disclosure, the kit may comprise a plurality of oligonucleotides configured for determining whether the recipient subject has at least one single nucleotide polymorphism in a gene locus or a plurality of gene loci, wherein the gene locus, or the plurality of gene loci, is selected from the group consisting of: CYP2C8, TUBB, or a combination of CYP2C8 and TUBB, and correlating the identity of the single nucleotide polymorphism or a plurality of single nucleotide polymorphisms to a Toxicity Index of the recipient subject to a taxane.

In one embodiment of the disclosure the kit comprises at least one oligonucleotide selected from the group of oligonucleotides consisting of the nucleotide sequences according to SEQ ID NOs: 3-44.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

### Description of Toxicity Index

Recipient subject-specific characteristics, medical and treatment histories, doses of experimental agents, and graded toxicities were extracted from case report forms. Toxicity data were represented for each subject as a set of treatment-attributable adverse events, each graded (0, 1, 2, 3, or 4) according to NCI Common Toxicity Criteria (CTC). The term “toxicity profile” as used herein refers to the collection of graded toxicities observed for an individual recipient subject.

To include all trials in the same analysis, a common definition of dose limiting toxicity (DLT) was adopted. Specifically, DLT was defined as occurring whenever any one of the following was observed: grade 4 hematological toxicity (grade 4 neutropenia or thrombocytopenia of any duration); a grade 3 or 4 non-hematological toxicity (excluding nausea, vomiting, and alopecia); febrile neutropenia of any grade.

To identify risk factors and other pre-trial characteristics of interest, several ways of summarizing the toxicity profile of a given recipient subject into a single measure of overall severity were examined as described, for example in Rogatko et al., (2004) Clin. Cancer Res. 10:4645-4651, incorporated herein by reference in its entirety. A summary measure that takes into account all observed toxicity grades rather than just the most severe one was used. The summary measure, the Toxicity Index (TI), is computed according to the following algorithm:
(a) recode each hematologic toxicity as the maximum of 0 and the observed grade minus one (so that a grade 3 toxicity is dose limiting irrespective of whether or not it is hematologic).
(b) Let the toxicity grades in a subject's recoded toxicity profile be represented in descending order by the sequence X₁≧X₂≧ . . . ≧Xₙ
(c) Calculate the subject's TI score as the weighted sum of the ordered toxicity grades:

$$TI = {\sum }_{i = 1}^{n} {w}_{i} {X}_{i} ,$$

where the weights are given by

$${w}_{i} = {\prod }_{j = 1}^{i - 1} {( {X}_{j} + 1 )}^{- 1} ,$$

or

$$TI = {X}_{1} + \frac{{X}_{2}}{1 + {X}_{1}} + \frac{{X}_{3}}{( 1 + {X}_{1} ) ( 1 + {X}_{2} )} + … + \frac{{X}_{n}}{( 1 + {X}_{1} ) … ( 1 + {X}_{n - 1} )} .$$

For example, a subject exhibiting two grade 3 toxicities would have a score of: TI=3+¾=3.75

Similarly, a subject exhibiting one grade 3 and ten grade 2 toxicities will have a score of: TI=3+ 2/4+2/(3.4)+2/(3².4)+ . . . +2/(3⁹.4).

A TI has the following properties:

Any score greater than or equal to 3 corresponds to a DLT according to the definition given above, and the maximum toxicity grade is the integer part of the final score. For example, TI=3.0 indicates a single grade 3 toxicity, whereas a score of 3.5 indicates that the recipient subject experienced at least one grade 3 toxicity plus additional toxicity. Hence, the TI preserves the information given by both the highest toxicity grade and the indicator of DLT. All toxicity grades are taken into account, though lower grades will contribute less to the final score.

The score is a number between 0 and 5.

As shown above, a large number of toxicities of the same grade will generate a TI score just slightly less than that generated by a single toxicity of the next higher grade.

### Example 2

### Genotyping Assay

The GENOMELAB SNPSTREAM™ (Beckman) was utilized to accommodate up to 92,000 genotypes per day. This automated, multiplexed system can process up to 48 SNPs in each well of an arrayed 384-well plate. The SNPSTREAM™ operates at a consistent cost per genotype or per sample, regardless of run throughput, allowing users to cost-effectively zero in on targets with low-throughput studies as well as conduct high-throughput analyses.

The SNPSTREAM™ Genotyping System features a protocol with three steps. The technology is based on single-based primer extension technology. The system's high sensitivity delivers results using as little as 2 ng of genomic DNA. SNPWARE™ reagent kits were used. The kits included ready-to-use reagents, all performed in the original PCR plate according to the manufacturer's instructions, and incorporated herein by reference in its entirety.

Multiplexed reactions were spatially resolved on a SNPWARE™ Tag Array plate. Each of the 384 wells in a plate contained 16 or 52 unique oligonucleotides of a known sequence, or tag. Each tag on the plate was complementary to one of the tags of the 12 or 48 extension primers, plus four controls to ensure accuracy. After transfer to the Tag Array plate, each SNP was hybridized to its complementary tag. SNPs were then identified by their position in the well.

The process involved a multiplex PCR followed by an annealing reaction of the oligonucleotide to the specific PCR product. Next the oligonucleotide was extended by a single base which was fluorescently labeled with a terminating NTP and finally detected by two-colored fluorescence specific for the allele.

### Example 3

Specific Primers and PCR Conditions Used for Genotyping CYP2C8 (rs1058932) and TUBB (rs3132584)

Primer sequences used were (PCRU and PCRL denote PCR amplification primers-forward and reverse primers, respectively: SNPU or SNPL designates a polymorphism-specific primer for single-base extension assays):

The nucleotide sequences from the genes CYP2C8 and TUBB2 flanking SNPs correlating to taxane toxicity are shown in FIGS. 3 and 4.

TABLE 1A

| | Gene | | | rs | |
|---|---|---|---|---|---|
| Gene | Polymorphism | SNP ID | Gene_TI | Number | Sequence Name |
| MAPT | MAPT-H2 | 17652121-TCU | MAPT_H2 | 17652121 | 17652121-TC-PCRU |
| | | | | | 17652121-TC-PCRL |
| | | | | | 17652121-TC-SNPU |
| MAPT | MAPT-H1/H2_1 | 754512-TAL | MAPT_H1_H21 | 754512 | 754512-TA-PCRU |
| | | | | | 754512-TA-PCRL |
| | | | | | 754512-TA-SNPL |
| MAPT | MAPT-H1 | 3785883-AGU | MAPT_H13 | 3785883 | 3785883-AG-PCRU |
| | | | | | 3785883-AG-PCRL |
| | | | | | 3785883-AG-SNPU |
| MAPT | MAPT-H1/H2 | 767058-AGU | MAPT_H1_H22 | 767058 | 767058-AG-PCRU |
| | | | | | 767058-AG-PCRL |
| | | | | | 767058-AG-SNPU |
| TUBB | TUBB | 8233-AGU | TUBB1 | 8233 | 8233-AG-PCRU |
| | | | | | 8233-AG-PCRL |
| | | | | | 8233-AG-SNPU |
| CYP2C8 | CYP2C8*2/*3 | 1058932-TCU | CYP2C8_2_{——}31 | 1058932 | 1058932-TC-PCRU |
| | | | | | 1058932-TC-PCRL |
| | | | | | 1058932-TC-SNPU |
| MAPT | MAPT-H1 | 242557-AGL | MAPT_H14 | 242557 | 242557-AG-PCRU |
| | | | | | 242557-AG-PCRL |
| | | | | | 242557-AG-SNPL |
| TUBB | TUBB | 3132584-CAU | TUBB2 | 3132584 | 3132584-CA-PCRU |
| | | | | | 3132584-CA-PCRL |
| | | | | | 3132584-CA-SNPU |
| MAPT | MAPT-H1/H2 | 2055794-AGU | MAPT_H1_H23 | 2055794 | 2055794-AG-PCRU |
| | | | | | 2055794-AG-PCRL |
| | | | | | 2055794-AG-SNPU |
| CYP3A5 | CYP3A5*3A | 776746-AGU | CYP3A5_3A | 776746 | 776746-AG-PCRU |
| | | | | | 776746-AG-PCRL |
| | | | | | 776746-AG-SNPU |
| CYP2C8 | CYP2C8*1C | 17110453-CAU | CYP2C8_1C | 17110453 | 17110453-CA-PCRU |
| | | | | | 17110453-CA-PCRL |
| | | | | | 17110453-CA-SNPU |
| TUBB | TUBB | 25527-TCU | TUBB3 | 25527 | 25527-TC-PCRU |
| | | | | | 25527-TC-PCRL |
| | | | | | 25527-TC-SNPU |
| CYP3A5 | CYP3A5*3A | 15524-TCU | CYP3A5_3A1 | 15524 | 15524-TC-PCRU |
| | | | | | 15524-TC-PCRL |
| | | | | | 15524-TC-SNPU |
| TUBB | TUBB | 1061397-TCU | TUBB4 | 1061397 | 1061397-TC-PCRU |
| | | | | | 1061397-TC-PCRL |
| | | | | | 1061397-TC-SNPU |

TABLE 1B

| | |
|---|---|
| Sequence Name | Sequence |

| | |
|---|---|
| 17652121-TC-PCRU | AAGAGCCGCCTGCAGACA |
| | SEQ ID NO: 9 |
| 17652121-TC-PCRL | GCTGGTGCTTCAGGTTCTC |
| | SEQ ID NO:10 |
| 17652121-TC-SNPU | TACCTATGACCAGCAAGCACCCGTGCCCATGCCA |
| | GACCTGAAGAA |
| | SEQ ID NO: 11 |
| 754512-TA-PCRU | TAAAAGCAAGACAGTTATTGTTTACTAGA |
| | SEQ ID NO: 12 |
| 754512-TA-PCRL | TTTTGTTTAGTGAACTTTTATCACCA |
| | SEQ ID NO: 13 |
| 754512-TA-SNPL | ACCGCACTAAGCAATGTATCTGGGATGTGACTTT |
| | CCAGAATGTTT |
| | SEQ ID NO: 14 |
| 3785883-AG-PCRU | TTGCTCAGCGATATTGTCAC |
| | SEQ ID NO: 15 |
| 3785883-AG-PCRL | GACACCCTCCGGGTCCAT |
| | SEQ ID NO: 16 |
| 3785883-AG-SNPU | CACTACATACGACCGCAGAATGACAAGGCACTGT |
| | CACCACTGGGC |
| | SEQ ID NO: 17 |
| 767058-AG-PCRU | TGAGGCAAGGAGGGGCTG |
| | SEQ ID NO: 18 |
| 767058-AG-PCRL | TAGAAGACAAACCTGGTCAGAG |
| | SEQ ID NO: 19 |
| 767058-AG-SNPU | CCATAACAACTTACCAGCCAGCTTCCTGAGCTCT |
| | CCGAGCTGCT |
| | SEQ ID NO: 20 |
| 8233-AG-PCRU | TTCTTAATCCCCACCTTTTCTT |
| | SEQ ID NO: 21 |
| 8233-AG-PCRL | AAAAGGAAGAAATGAGATGTTGC |
| | SEQ ID NO: 22 |
| 8233-AG-SNPU | AGACCGACAAGCAATCTACAAAGAATGAACACCC |
| | CTGACTCTGGA |
| | SEQ ID NO: 23 |
| 1058932-TC-PCRU | ATACCAGATCTGCTTCATCCC |
| | SEQ ID NO: 3 |
| 1058932-TC-PCRL | AAGATTTGATGAGAGGTCAGAGAA |
| | SEQ ID NO: 4 |
| 1058932-TC-SNPU | AATAAGCTCACCACCGTCAAGAAGAATGCTAGCC |
| | CATCTGGCTGC |
| | SEQ ID NO: 7 |
| 242557-AG-PCRU | TAGAGTGTACGTTTCTTCTTCCTT |
| | SEQ ID NO: 24 |
| 242557-AG-PCRL | TGGTCCCGTGACACCCTG |
| | SEQ ID NO: 25 |
| 242557-AG-SNPL | CAACAAGACATAACAACGCAACAGAGCCAAAACC |
| | GTGTCCTGGTG |
| | SEQ ID NO: 26 |
| 3132584-CA-PCRU | CAAAGGCAAATGCTAGCTACA |
| | SEQ ID NO: S |
| 3132584-CA-PCRL | TTTTTACAAGGAAAAATCCAGGT |
| | SEQ ID NO: 6 |
| 3132584-CA-SNPU | GCAACATAAGACCGCTCAACAGGGGCAAATCTTG |
| | ATTAAGGATAG |
| | SEQ ID NO: 8 |
| 2055794-AG-PCRU | TATAGAACACAATTCCTTCAGAGTATAATC |
| | SEQ ID NO: 27 |
| 2055794-AG-PCRL | TATTGTGTGTAGCATACGTCCTTTA |
| | SEQ ID NO: 28 |
| 2055794-AG-SNPU | GATCCATCAACAGACATCACCCTTTGTCATTGAA |
| | GTGTATAATTT |
| | SEQ ID NO: 29 |
| 776746-AG-PCRU | TGGCATAGGAGATACCCAC |
| | SEQ ID NO: 30 |
| 776746-AG-PCRL | TTCATATGATGAAGGGTAATGTG |
| | SEQ ID NO: 31 |
| 776746-AG-SNPU | CAACAATACGAGCCAGCAAGCTTTAAAGAGCTCT |
| | TTTGTCTTTCA |
| | SEQ ID NO: 32 |
| 17110453-CA-PCRU | AAACACTGAAGTAAATGATTCTATGTTAGA |
| | SEQ ID NO: 33 |
| 17110453-CA-PCRL | GCATTACAATGTACATTTTTTATACACA |
| | SEQ ID NO: 34 |
| 17110453-CA-SNPU | AGCAAGACCACCTAGACCAGTTCTCAGATTAATG |
| | ACCAGTTGGGA |
| | SEQ ID NO: 35 |
| 25527-TC-PCRU | AAAAGTTAGGAGATGATTGTTGTATTG |
| | SEQ ID NO: 36 |
| 25527-TC-PCRL | TATCTTCTTTCTCCTTCACTGTGATAT |
| | SEQ ID NO: 37 |
| 25527-TC-SNPU | CCAGATCCTCACCATGTAAGTACAGAAATGTGTT |
| | CTGAAATCTAA |
| | SEQ ID NO: 38 |
| 15524-TC-PCRU | ATTGTTCTAAAGGTGGATTCAAGA |
| | SEQ ID NO: 39 |
| 15524-TC-PCRL | ATTAGATTAAGCCCATCTTTATTTCA |
| | SEQ ID NO: 40 |
| 15524-TC-SNPU | ACAATCAACATACGAACAGCAGTGGAGAATGAGT |
| | TATTCTAAGGA |
| | SEQ ID NO: 41 |
| 1061397-TC-PCRU | ATCCCATTTAGAACCAACCAG |
| | SEQ ID NO: 42 |
| 1061397-TC-PCRL | TACCCACTACCTTCTACCATTTT |
| | SEQ ID NO: 43 |
| 1061397-TC-SNPU | TACAAGCACGCACTAGACTGCTGAAAACACATGT |
| | AGATAATGGC |
| | SEQ ID NO: 44 |

### Example 4

For each SNP, the -PCRU and -PCRL primers were first used to amplify a relevant DNA fragment surrounding the target SNP site. The components per reaction were: H₂O, 3.0 μl; 10× PCR Buffer II, 0.5 μl; MgCl₂ (25 mM), 1.0 μl; dNTPs (2.5 mM each), 0.18 μl; PCR Primer Pool (2.5 μM each), 0.1 μl; AMPLITAQ GOLD™ (5 U/μl), 0.125 μl for a total volume of 5.0 μl per reaction.

The reaction conditions were Step 1: 94° C. for 1 min; Step 2: 94° C for 30 sec; Step 3: 55° C. for 30 sec; Step 4: 72° C. for 1 min; and Step 5: return to step 2 for 39 times, and finally hold at 4° C.

Primer -SNPU or SNPL was then used for single-base extension reaction. The thermocycler program was as follows: Step 1: 96° C. for 3 min; Step 2: 94° C. for 20 sec; Step 3: 40° C. for 11 sec; and Step 5: return to step 2 59 times, and finally to hold at 4° C.

### Example 5

### Statistical Analysis

Thirty (30) recipient subjects already receiving treatment with paclitaxel were screened for the presence of SNPs in the CYP2C8 and TUBB loci. Clinical data from each of the recipient subjects was used to first calculate a Toxicity Index (TI) value for each individual according to the method presented in Example 1, above. The genotype for each recipient subject with regard to the SNPs of CYP2C8 and TUBB was determined using SNPWARE™ and single base extension according to the methods of Example 4, above.

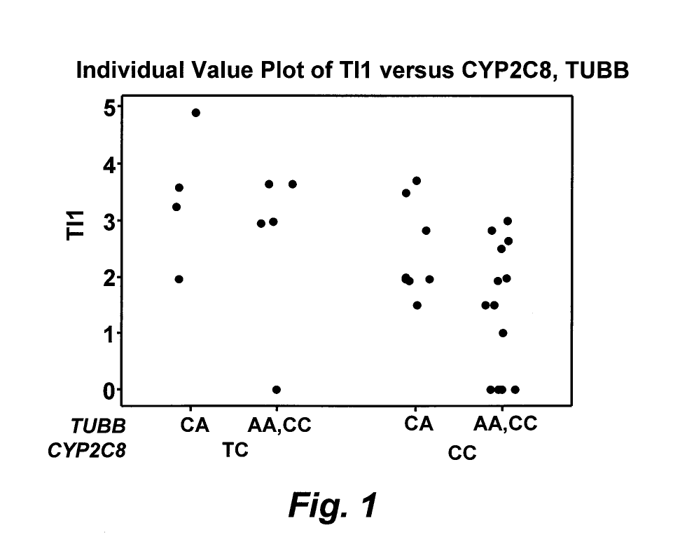

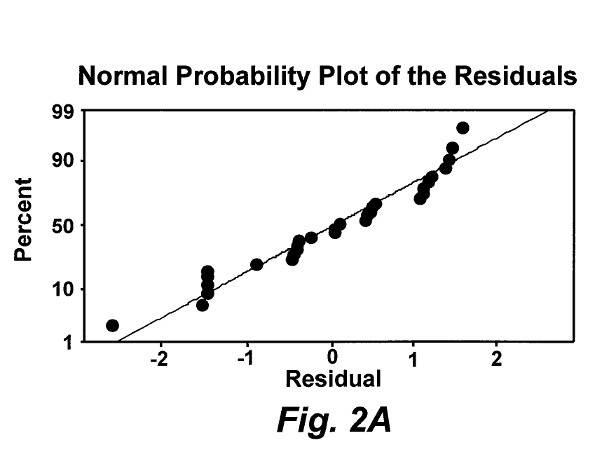

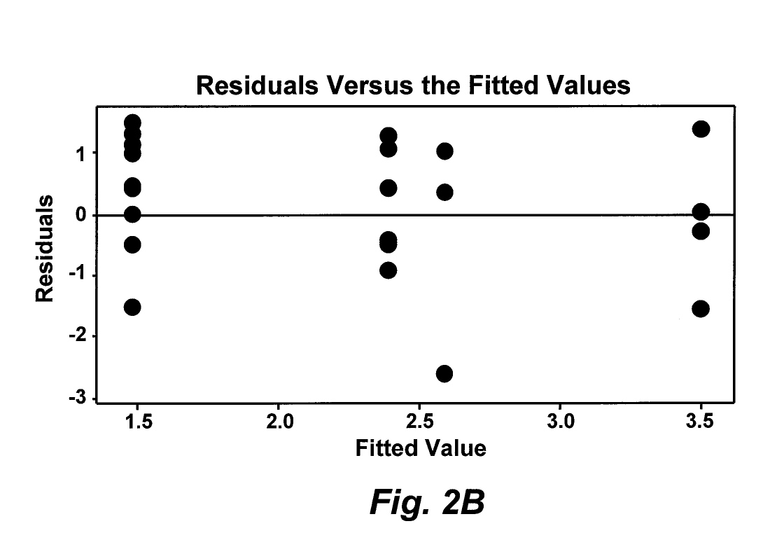

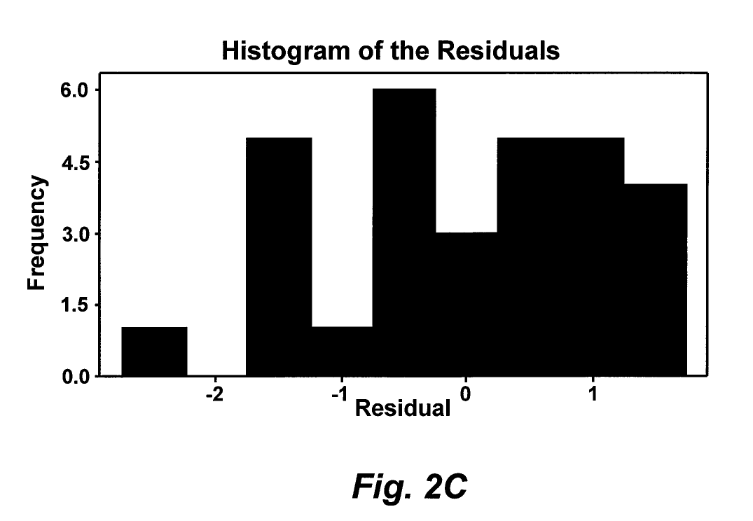

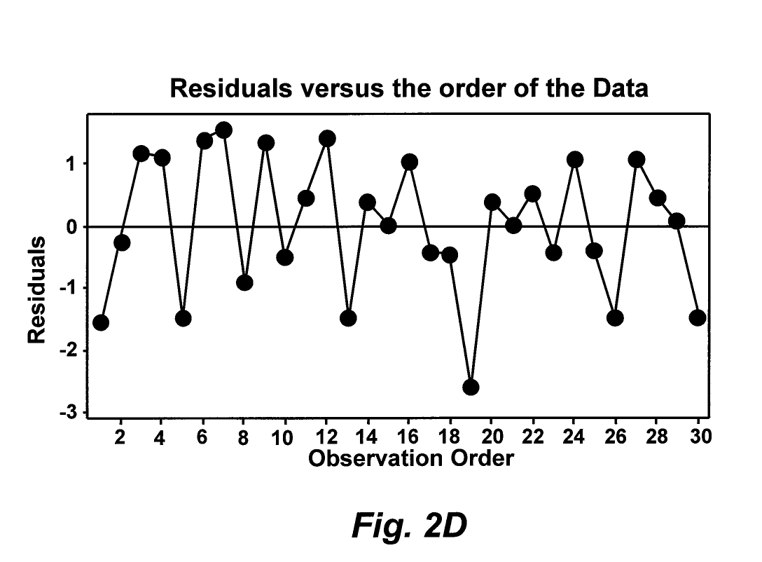

For the CYP2C8 locus, two genotypes for the rsl 058932 SNP (as shown in FIG. 1) were considered: heterozygous TC and homozygous CC. For the TUBB locus (SNP rs3132584), the heterozygous SNP CA, and the combined homozygous AA and CC variants, were considered.

Regression Analysis: TI1 versus CYP2C8_{—}2_{—}31, TUBB2

The regression equation is:

TI1=5.52−1.11 CYP2C8_{—}2_{—}31−0.913 TUBB2

TABLE 2

| Predictor | Coef | SE Coef | T | P |
|---|---|---|---|---|
| Constant | 5.524 | 1.009 | 5.47 | 0.000 |
| CYP2C8_2_31 | −1.1100 | 0.4498 | −2.47 | 0.020 |
| TUBB2 | −0.9135 | 0.4207 | −2.17 | 0.039 |

S = 1.12694 R-Sq = 29.8% R-Sq(adj) = 24.6%

Analysis of Variance

| Source | DF | SS | MS | F | P |
|---|---|---|---|---|---|
| Regression | 2 | 14.581 | 7.291 | 5.74 | 0.008 |
| Residual Error | 27 | 34.290 | 1.270 | | |
| Total | 29 | 48.871 | | | |

| Source | DF | Seq SS |
|---|---|---|
| CYP2C8_2_31 | 1 | 8.594 |
| TUBB2 | 1 | 5.987 |

### Example 6

A preliminary data analysis focused on the relationship between the Toxicity Index and the recipient subjects' demographic/genetic information. Toxicity indices at cycle 1 to cycle 5 were considered in the analysis since more than 50% of the recipient subjects showed Toxicity Index during those cycles. Univariate linear regression analysis was conducted first to find significant covariates in predicting the Toxicity Index in one of five cycles, then multivariable analysis was used to determine the best model in fitting the data. A mixed model for repeated measurement was implemented in modeling Toxicity Index in the first five cycles, and also in all cycles. Autoregressive covariance structure was used in the mixed model. SAS and Minitab were used in the analysis.

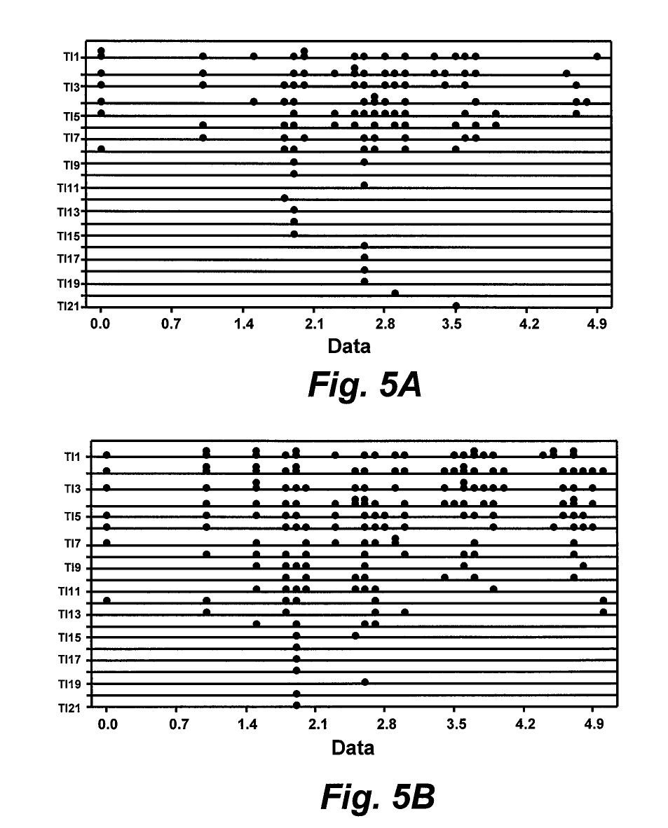

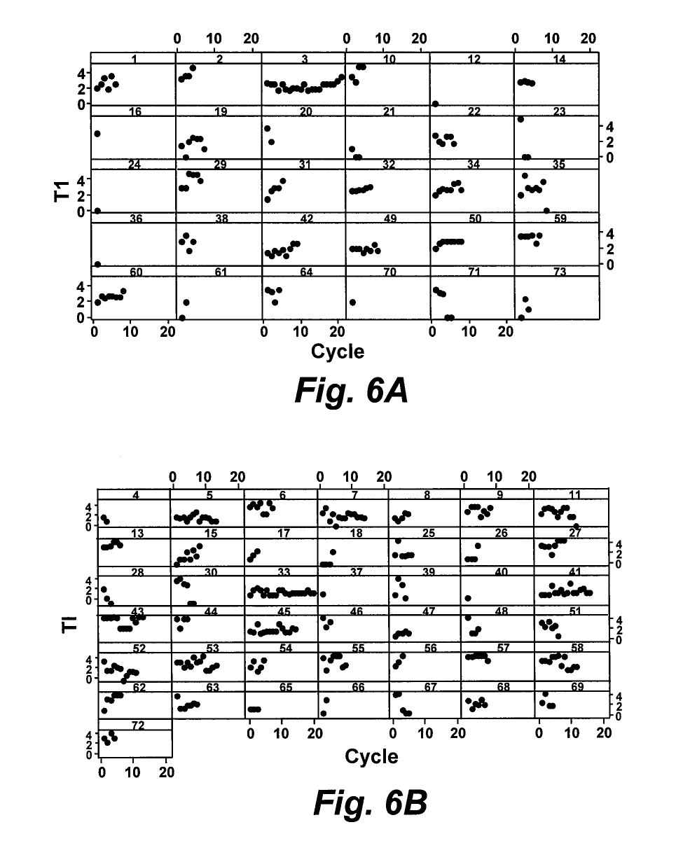

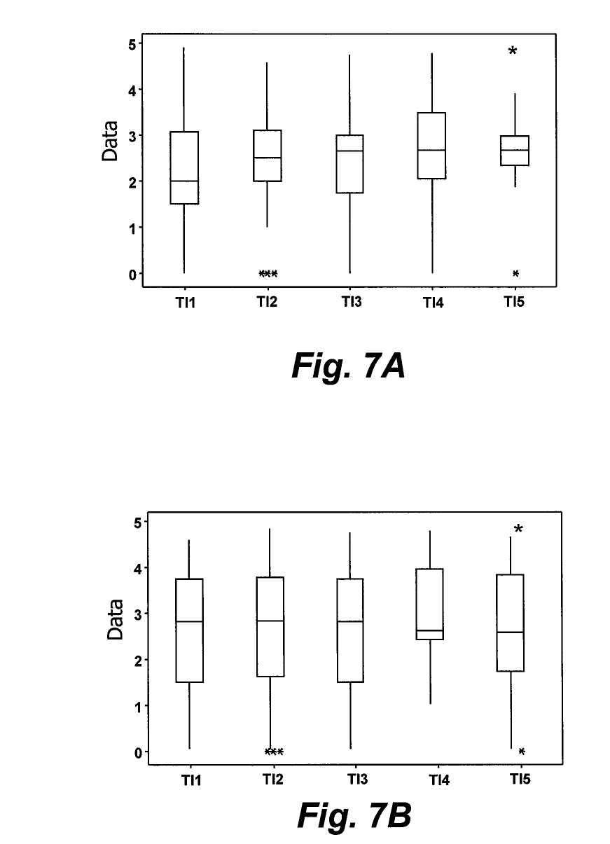

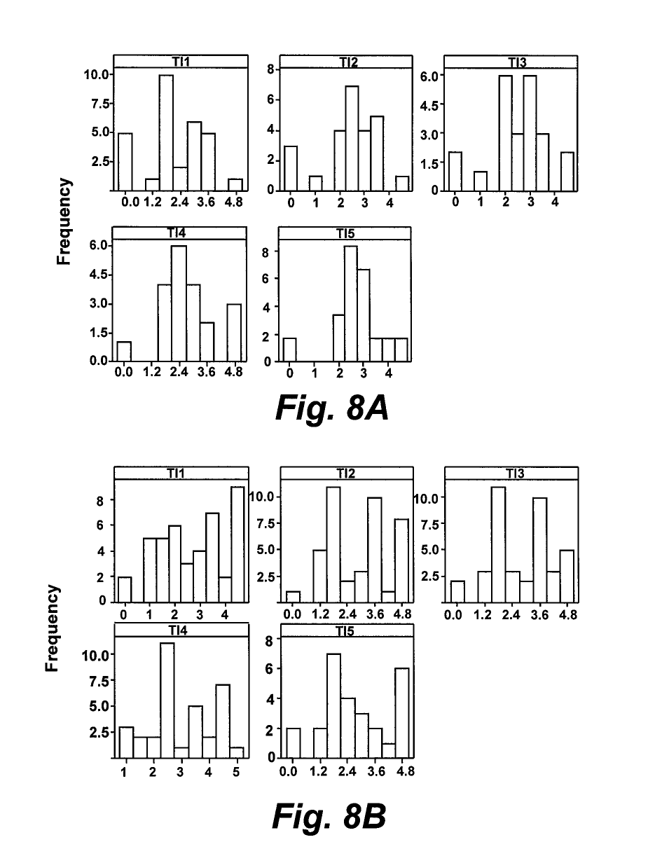

The descriptive statistics of demographic variables was shown in Table 3. The distributions of the Toxicity Index at each cycle and for each recipient subject were displayed in FIGS. 5A-6B. The frequency table of the Toxicity Index in each cycle is shown in Table 4.

TABLE 3
Descriptive statistics of demographic variables

| | | Paclitaxel | | Docetaxel | | Total | |
|---|---|---|---|---|---|---|---|
| Variable | Value | N (percent) | | N (percent) | | N (percent) | |
| Sex | Female | 16 | (53.3%) | 0 | | 16 | (21.9%) |
| | Male | 14 | (46.7%) | 43 | (100%) | 57 | (78.1%) |
| Race | White | 28 | (93.3%) | 38 | (88.4%) | 66 | (90.4%) |
| | Non- | 2 | (6.7%) | 5 | (11.6%) | 7 | (9.6%) |
| | white | | | | | | |
| Site | Prostate | 0 | | 38 | (88.4%) | 38 | (52.1%) |
| | Gland | | | | | | |
| | Others | 30 | (100%) | 5 | (11.6%) | 35 | (47.9%) |
| Stage | T1 | | | 4 | (11.4%) | 4 | (7.1%) |
| | T2 | 6 | (28.6%) | 11 | (31.4%) | 17 | (30.4%) |
| | T3 | 4 | (19.1%) | 8 | (22.9%) | 12 | (21.4%) |
| | T4 | 7 | (33.3%) | 0 | | 7 | (12.5%) |
| | TX | 4 | (19.1%) | 12 | (34.3%) | 16 | (28.6%) |
| Grade | Poorly | 16 | (53.3%) | 7 | (16.3%) | 23 | (31.5%) |
| | Dif | | | | | | |
| | Others | 14 | (46.7%) | 36 | (83.7%) | 50 | (68.5%) |
| Total | | 30 | | 43 | | 73 | |
| Age | Mean | 58.8 | (11.4) | 65.9 | (9.0) | | |
| | (std.) | | | | | | |
| Received | Mean | 287.3 | (126.0) | 147.3 | (23.6) | | |
| | (std.) | | | | | | |
| Dose | | | | | | | |

TABLE 4
Frequency table of TI in each cycle

| | Paclitaxel | | | Docetaxel | | |
|---|---|---|---|---|---|---|
| Cycle | N | Mean | Std | N | Mean | Std |
| TI1 | 30 | 2.175 | 1.298 | 43 | 2.727 | 1.395 |
| TI2 | 25 | 2.399 | 1.155 | 41 | 2.840 | 1.383 |
| TI3 | 23 | 2.491 | 1.196 | 39 | 2.737 | 1.361 |
| TI4 | 20 | 2.787 | 1.176 | 34 | 3.058 | 1.208 |
| TI5 | 15 | 2.696 | 1.048 | 27 | 2.669 | 1.457 |
| TI6 | 13 | 2.580 | 0.849 | 23 | 2.875 | 1.346 |
| TI7 | 8 | 2.529 | 0.926 | 17 | 2.646 | 1.183 |
| TI8 | 7 | 2.206 | 1.139 | 15 | 2.804 | 1.273 |
| TI9 | 2 | 2.292 | 0.501 | 10 | 2.438 | 1.027 |
| TI10 | 1 | 1.875 | — | 10 | 2.623 | 1.024 |
| TI11 | 1 | 2.583 | — | 10 | 2.297 | 0.732 |
| TI12 | 1 | 1.750 | — | 9 | 2.090 | 1.360 |
| TI13 | 1 | 1.875 | — | 7 | 2.404 | 1.300 |
| TI14 | 1 | 1.875 | — | 4 | 2.171 | 0.578 |
| TI15 | 1 | 1.875 | — | 3 | 2.083 | 0.361 |
| TI16 | 1 | 2.583 | — | 2 | 1.875 | 0.000 |
| TI17 | 1 | 2.583 | — | 1 | 1.875 | — |
| TI18 | 1 | 2.583 | — | 1 | 1.875 | — |
| TI19 | 1 | 2.583 | — | 1 | 2.625 | — |
| TI20 | 1 | 2.944 | — | 1 | 1.875 | — |
| TI21 | 1 | 3.469 | — | 1 | 1.875 | — |

Univariate linear regression analysis was applied to the Toxicity Index in the first five cycles since more than 50% of the recipient subjects showed Toxicity Index in those cycles. Covariates in the analysis included recoded binary demographic variables (sex, race, site, grade, stage) and recoded binary genetic variables.

TABLE 5
Univariate analysis result

| Regimen | Dep. Var. | Indep. Var. | Para. Est. | P-value | Comments |
|---|---|---|---|---|---|
| Paclitaxel | TI1 | TUBB1 | 0.975 | 0.042 | AG = 1, AA/GG = 0. |
| | TI1 | CYP2C8_2_31 | −1.168 | 0.021 | CC = 1, TC = 0. |
| | TI1 | MAPT_H14 | 1.151 | 0.016 | TC = 1, CC/TT = 0. |
| | TI1 | TUBB2 | 0.975 | 0.042 | CA = 1, CC/AA = 0. |
| | TI2 | TUBB3 | −1.062 | 0.047 | CC = 1, TC/TT = 0. |
| | TI2 | TUBB4 | −1.185 | 0.037 | CC = 1, TC/TT = 0. |
| | TI3 | MAPT_H14 | 1.236 | 0.012 | TC = 1, CC/TT = 0. |
| | TI4 | CYP2C8_1C | −1.267 | 0.033 | AA = 1, CA = 0. |
| | TI5 | CYP3A5_3A | 1.477 | 0.022 | GG = 1, AG = 0. |
| | TI5 | CYP3A5_3A1 | 1.477 | 0.022 | TT = 1, TC = 0. |
| Docetaxel | TI3 | Site | 1.962 | 0.046 | Prostate Gland = 1, |
| | | | | | Others = 0. |
| | TI3 | Grade | −1.609 | 0.006 | Poorly Dif = 1, |
| | | | | | Others = 0. |
| | TI3 | MAPT_H2 | −1.166 | 0.006 | TT = 1, CC/TC = 0. |
| | TI3 | MAPT_H1_H21 | −1.107 | 0.010 | AA = 1, TA/TT = 0. |
| | TI3 | MAPT_H13 | −1.245 | 0.013 | GG = 1, AG = 0. |
| | TI3 | MAPT_H1_H22 | −1.166 | 0.006 | AA = 1, AG/GG = 0. |
| | TI3 | MAPT_H1_H23 | −1.107 | 0.010 | GG = 1, AA/AG = 0. |
| | TI4 | MAPT_H2 | −0.900 | 0.029 | TT = 1, CC/TC = 0. |
| | TI4 | MAPT_H1_H21 | −0.953 | 0.023 | AA = 1, TA/TT = 0. |
| | TI4 | MAPT_H1_H22 | −0.900 | 0.029 | AA = 1, AG/GG = 0. |
| | TI4 | MAPT_H1_H23 | −0.953 | 0.023 | GG = 1, AA/AG = 0. |
| | TI4 | CYP3A5_3A | −1.079 | 0.045 | AG = 1, GG/AA = 0. |
| | TI4 | CYP3A5_3A1 | −1.079 | 0.045 | TC = 1, CC/TT = 0. |
| | TI5 | CYP3A5_3A | −1.739 | 0.024 | AG = 1, GG/AA = 0. |
| | TI5 | CYP3A5_3A1 | −1.739 | 0.024 | TC = 1, CC/TT = 0. |

Multivariable regression analysis was used to find the best model in predicting the Toxicity Index in each of the first five cycles. The result is listed in Table 6.

TABLE 6
Multivariable analysis result

| | Dep. | | Para. | | | |
|---|---|---|---|---|---|---|
| Regimen | Var. | Indep. Var. | Est. | SE | P-value | Comments |
| Paclitaxel | TI1 | Intercept | 2.587 | 0.420 | 0.000 | |
| | | CYP2C8_2_31 | −1.110 | 0.450 | 0.020 | CC = 1, TC = 0. |
| | | TUBB2 | 0.914 | 0.421 | 0.039 | CA = 1, CC/AA = 0. |
| | | Root MSE = 1.127 | R² = 0.298 | R²_{adj} = 0.246 | | |
| | TI2 | Intercept | 3.347 | 0.479 | 0.000 | |
| | | TUBB4 | −1.185 | 0.536 | 0.037 | CC = 1, TC/TT = 0. |
| | | Root MSE = 1.072 | R² = 0.175 | R²_{adj} = 0.140 | | |
| | TI3 | Intercept | 2.008 | 0.280 | 0.000 | |
| | | MAPT_H14 | 1.236 | 0.448 | 0.012 | TC = 1, CC/TT = 0. |
| | | Root MSE = 1.049 | R² = 0.266 | R²_{adj} = 0.231 | | |
| | TI4 | Intercept | 3.738 | 0.475 | 0.000 | |
| | | CYP2C8_1C | −1.267 | 0.548 | 0.033 | AA = 1, CA = 0. |
| | | Root MSE = 1.061 | R² = 0.229 | R²_{adj} = 0.186 | | |
| | TI5 | Intercept | 1.514 | 0.510 | 0.011 | |
| | | CYP3A5_3A | 1.477 | 0.571 | 0.022 | GG = 1, AG = 0. |
| | | Root MSE = 0.883 | R² = 0.341 | R²_{adj} = 0.290 | | |
| Docetaxel | TI3 | Intercept | −0.533 | 0.926 | 0.568 | |
| | | Site | 2.365 | 0.842 | 0.001 | Prostate Gland = 1, |
| | | | | | | Others = 0. |
| | | MAPT_H13 | 1.408 | 0.442 | 0.003 | GG = 1, AG = 0. |
| | | Root MSE = 1.150 | R² = 0.313 | R²_{adj} = 0.274 | | |
| | TI4 | Intercept | 3.561 | 0.294 | 0.000 | |
| | | MAPT_H2 | −0.900 | 0.393 | 0.029 | TT = 1, CC/TC = 0. |
| | | Root MSE = 1.137 | R² = 0.141 | R²_{adj} = 0.114 | | |
| | TI5 | Intercept | 2.936 | 0.279 | 0.000 | |
| | | CYP3A5_3A | −1.739 | 0.726 | 0.024 | AG = 1, GG/AA = 0. |
| | | Root MSE = 1.340 | R² = 0.187 | R²_{adj} = 0.154 | | |

A mixed model for repeated measurements was implemented to find the best model in fitting the longitudinal data. Autoregressive covariance structure was used in the mixed model. Toxicity Indexes in the first five cycles were included in the analysis.

For the paclitaxel, MAPT_H14 was a significant fixed effect (p=0.012) in predicting the longitudinal Toxicity Index in five cycles.

Solution for Fixed Effects

| | | Standard | | | |
|---|---|---|---|---|---|
| Effect | Estimate | Error | DF | t Value | Pr > \|t\| |
| Intercept | 2.0279 | 0.2069 | 28 | 9.80 | <.0001 |
| COL18_3 | 0.8918 | 0.3309 | 28 | 2.70 | 0.0118 |

For the docetaxel, grade (prostate gland=1, others=0) and MAPT_H1 (TT=1, CC=0) were significant fixed effects in predicting the longitudinal Toxicity Index in five cycles.

Solution for Fixed Effects

| | | Standard | | | |
|---|---|---|---|---|---|
| Effect | Estimate | Error | DF | t Value | Pr > \|t\| |
| Intercept | 1.9361 | 0.4292 | 38 | 4.51 | <.0001 |
| Grade 2 | −1.0426 | 0.3881 | 38 | −2.69 | 0.0106 |
| COL6_2 | 1.1407 | 0.4568 | 38 | 2.50 | 0.0170 |

To model Toxicity Indexes in all 21 cycles, a mixed model was used to find the best model. For the paclitaxel, Received dose and MAPT_H14 were significant fixed effects in predicting the longitudinal Toxicity Index in all cycles.

Solution for Fixed Effects

| | | Standard | | | |
|---|---|---|---|---|---|
| Effect | Estimate | Error | DF | t Value | Pr > \|t\| |
| Intercept | 1.4233 | 0.3230 | 27 | 4.41 | 0.0001 |
| Received Dose2 | 0.002363 | 0.000991 | 27 | 2.38 | 0.0244 |
| COL18_3 | 0.6989 | 0.2573 | 27 | 2.72 | 0.0114 |

For the Docetaxel, grade (prostate gland=1, others=0) and MAPT_H1 (TT=1, CC=0) were significant fixed effects in predicting the longitudinal Toxicity Index in all cycles.

Solution for Fixed Effects

| | | Standard | | | |
|---|---|---|---|---|---|
| Effect | Estimate | Error | DF | t Value | Pr > \|t\| |
| Intercept | 1.8615 | 0.3310 | 38 | 5.62 | <.0001 |
| Grade 2 | −0.9840 | 0.3060 | 38 | −3.22 | 0.0027 |
| COL6_2 | 1.1286 | 0.3547 | 38 | 3.18 | 0.0029 |

We claim:

1. A method of predicting the toxicity of a taxane to a recipient subject, comprising:
(a) isolating a nucleic acid sample from a recipient subject;
(b) determining a genotype of the recipient subject from the isolated nucleic acid sample, thereby determining the identity of at least one single nucleotide polymorphism within the genome of the recipient subject, wherein the identity of the at least one single nucleotide polymorphism is correlated to a Toxicity Index value associated with the exposure of a recipient subject to a taxane; and
(c) providing a prognostic determination of the level of toxicity of a taxane on the recipient subject, wherein the presence of at least one single nucleotide polymorphism within at least one gene locus correlates to a Toxicity Index value, wherein the Toxicity Index value provides a prognostic determination of the level of toxicity of a taxane on the recipient subject.

2. The method according to claim 1, wherein step (b) comprises determining the identity of a first single nucleotide polymorphism within a gene locus selected from the group consisting of: a CYP2C8 gene locus and a TUBB gene locus.

3. The method according to claim 1, wherein step (b) comprises determining the identity of a first single nucleotide polymorphism and a second single nucleotide polymorphism, wherein the first single nucleotide polymorphism is within a CYP2C8 gene locus, and the second single nucleotide polymorphism is within a TUBB gene locus.

4. The method according to claim 3, wherein: (a) the first single nucleotide polymorphism within a CYP2C8 gene locus has the GenBank SNP Accession No. rs1058932, and wherein if the recipient subject is heterozygous TC at the first single nucleotide polymorphism, the recipient subject has an elevated probability of susceptibility to a taxane toxicity; and (b) the second single nucleotide polymorphism within a TUBB gene locus has the GenBank SNP Accession No. 3132584, and wherein if the recipient subject is heterozygous CA at the first single nucleotide polymorphism, the recipient subject has an elevated probability of susceptibility to a taxane toxicity.

5. The method according to claim 1, wherein the step (b) of determining the genotype of a recipient subject comprises:
amplifying a first region of the nucleic acid isolated from the recipient subject using a first forward oligonucleotide primer and a first reverse oligonucleotide primer, wherein the amplified region of the nucleic acid from the recipient subject comprises a first single nucleotide polymorphism site, and wherein the first single nucleotide polymorphism site is associated with a Toxicity Index for a taxane administered to the recipient subject; and
determining the nucleotide identity of the first single nucleotide polymorphism site within the first amplified polynucleotide region, thereby determining the Toxicity Index for a taxane administered to the recipient subject.

6. The method according to claim 5, wherein the step (b) of determining the genotype of a recipient subject further comprises:
amplifying a second region of the nucleic acid isolated from the recipient subject using a second forward oligonucleotide primer and a second reverse oligonucleotide primer, wherein the amplified region of the nucleic acid from the recipient subject comprises a second single nucleotide polymorphism site, and wherein the second single nucleotide polymorphism site is associated with a Toxicity Index for a taxane administered to the recipient subject; and
determining the nucleotide identity of the second single nucleotide polymorphism site within the second amplified polynucleotide region, thereby determining the Toxicity Index for a taxane administered to the recipient subject.

7. The method according to claim 5, wherein the first forward oligonucleotide primer has a nucleotide sequence selected from the group consisting of SEQ ID NOs: 3, 5, 9, 12, 15, 18, 21, 24, 27, 30, 33, 36, 39, and 42.

8. The method according to claim 5, wherein the first reverse oligonucleotide primer has a nucleotide sequence selected from the group consisting of SEQ ID NOs: 4, 6, 10, 13, 16, 19, 22, 25, 28, 31, 34, 37, 40, and 43.

9. The method according to claim 5, wherein the first forward oligonucleotide primer has a nucleotide sequence according to SEQ ID NO: 3 and the first reverse oligonucleotide primer has a nucleotide sequence according to SEQ ID NO: 4.

10. The method according to claim 5, wherein the first forward oligonucleotide primer has a nucleotide sequence according to SEQ ID NO: 5 and the first reverse oligonucleotide primer has a nucleotide sequence according to SEQ ID NO: 6.

11. The method according to claim 5, wherein the step of identifying the nucleotide identity of the first single nucleotide polymorphism site is by single base extension from an oligonucleotide primer selectively hybridizing to the amplified region under high stringency conditions, and wherein the oligonucleotide primer has a nucleotide sequence selected from the group consisting of SEQ ID NOs: 7, 8, 11, 14, 17, 20, 23, 26, 29, 32, 35, 38, 41, and 44.

12. The method according to claim 6, wherein the step of identifying the nucleotide identity of the second single nucleotide polymorphism site is by single base extension from an oligonucleotide primer selectively hybridizing to the amplified region under high stringency conditions, and wherein the oligonucleotide primer has a nucleotide sequence selected from the group consisting of SEQ ID NOs: 7, 8, 11, 14, 17, 20, 23, 26, 29, 32, 35, 38, 41, and 44.

13. The method according to claim 11, wherein the first single nucleotide polymorphism site is within the CYP28C gene locus, and wherein the nucleotide identity of the single nucleotide polymorphism is determined by single base extension from an oligonucleotide primer selectively hybridizing to the amplified region under high stringency conditions.

14. The method according to claim 13, wherein the oligonucleotide primer has a nucleotide sequence according to SEQ ID NO: 7.

15. The method according to claim 5, wherein the second primer oligonucleotide has a nucleotide sequence according to SEQ ID NO: 5 and the second primer oligonucleotide has a nucleotide sequence according to SEQ ID NO: 6.

16. The method according to claim 13, wherein the first single nucleotide polymorphism site within the TUBB2 gene locus, and wherein the nucleotide identity of the single nucleotide polymorphism is determined by single base extension from an oligonucleotide primer selectively hybridizing to the amplified region under high stringency conditions.

17. The method according to claim 14, wherein the oligonucleotide primer has a nucleotide sequence according to SEQ ID NO: 8.

18. The method according to claim 6, wherein the first forward oligonucleotide primer has a nucleotide sequence according to SEQ ID NO: 3, the first reverse oligonucleotide primer has a nucleotide sequence according to SEQ ID NO: 4, the second primer oligonucleotide has a nucleotide sequence according to SEQ ID NO: 5, and the second primer oligonucleotide has a nucleotide sequence according to SEQ ID NO: 6, and wherein the first single nucleotide polymorphism site is within the CYP28C gene locus and the second single nucleotide polymorphism site is within the TUBB gene locus, and wherein the nucleotide identity of the first single nucleotide polymorphism is determined by single base extension from an oligonucleotide primer having the nucleotide sequence according to SEQ ID NO: 7, and wherein the nucleotide identity of the second single nucleotide polymorphism is determined by single base extension from an oligonucleotide primer having the nucleotide sequence according to SEQ ID NO: 8.

19. The method according to claim 1, wherein the Toxicity Index is determined for a taxane selected from the group consisting of: paclitaxel and docetaxel.

20. A kit for predicting a level of toxicity of a taxane to a recipient subject by determining whether the recipient subject has at least one single nucleotide polymorphism in a gene locus, wherein the identity of the single nucleotide polymorphism(s) is correlated to a Toxicity Index of the recipient subject to a taxane, thereby providing a prognostic determination of the toxicity of a taxane to the recipient subject, the kit comprising at least one vessel containing at least one primer oligonucleotide, and instructions for using the primer oligonucleotide to determine the identity of a single nucleotide polymorphism in at least one gene locus of a nucleic acid sample isolated from the recipient subject.

21. The kit according to claim 20, further comprising a plurality of oligonucleotides configured for determining whether the recipient subject has at least one single nucleotide polymorphism in a gene locus or a plurality of gene loci, wherein the gene locus, or the plurality of gene loci, is selected from the group consisting of: TUBB, CYP2C8, MAPT, CYP3A5, and a combination thereof, and correlating the identity of the single nucleotide polymorphism or a plurality of single nucleotide polymorphisms to a Toxicity Index of the recipient subject to a taxane.

22. The kit according to claim 20, further comprising a plurality of oligonucleotides configured for determining whether the recipient subject has at least one single nucleotide polymorphism in a gene locus or a plurality of gene loci, wherein the gene locus, or the plurality of gene loci, is selected from the group consisting of: CYP2C8, TUBB, or a combination of CYP2C8 and TUBB, and correlating the identity of the single nucleotide polymorphism or a plurality of single nucleotide polymorphisms to a Toxicity Index of the recipient subject to a taxane.

23. The kit according to claim 17, comprising at least one oligonucleotide selected from the group of oligonucleotides consisting of the nucleotide sequences according to SEQ ID NOs: 3-44.


SEQUENCE LISTING (44 sequences)

SEQ ID NO: 1 - DNA, 1924 bp, Artificial sequence
  CYP2C8 rs1058932 flanking genomic sequence
acatgtcaaagagacacacactaaattagcagggagtgttataaaaactttggagtgcaagctcacagctgtcttaataagaagagaaggcttcaatggaaccttttgtggtcctggtgctgtgtctctcttttatgcttctcttttcactctggagacagagctgtaggagaaggaagctccctcctggccccactcctcttcctattattggaaatatgctacagatagatgttaaggacatctgcaaatctttcaccaatttctcaaaagtctatggtcctgtgttcaccgtgtattttggcatgaatcccatagtggtgtttcatggatatgaggcagtgaaggaagccctgattgataatggagaggagttttctggaagaggcaattccccaatatctcaaagaattactaaaggacttggaatcatttccagcaatggaaagagatggaaggagatccggcgtttctccctcacaaccttgcggaattttgggatggggaagaggagcattgaggaccgtgttcaagaggaagctcactgccttgtggaggagttgagaaaaaccaaggcttcaccctgtgatcccactttcatcctgggctgtgctccctgcaatgtgatctgctccgttgttttccagaaacgatttgattataaagatcagaattttctcaccctgatgaaaagattcaatgaaaacttcaggattctgaactccccatggatccaggtctgcaataatttccctctactcattgattgtttcccaggaactcacaacaaagtgcttaaaaatgttgctcttacacgaagttacattagggagaaagtaaaagaacaccaagcatcactggatgttaacaatcctcgggactttatcgattgcttcctgatcaaaatggagcaggaaaaggacaaccaaaagtcagaattcaatattgaaaacttggttggcactgtagctgatctatttgttgctggaacagagacaacaagcaccactctgagatatggactcctgctcctgctgaagcacccagaggtcacagctaaagtccaggaagagattgatcatgtaattggcagacacaggagcccctgcatgcaggataggagccacatgccttacactgatgctgtagtgcacgagatccagagatacagtgaccttgtccccaccggtgtgccccatgcagtgaccactgatactaagttcagaaactacctcatccccaagggcacaaccataatggcattactgacttccgtgctacatgatgacaaagaatttcctaatccaaatatctttgaccctggccactttctagataagaatggcaactttaagaaaagtgactacttcatgcctttctcagcaggaaaacgaatttgtgcaggagaaggacttgcccgcatggagctatttttatttctaaccacaattttacagaactttaacctgaaatctgttgatgatttaaagaacctcaatactactgcagttaccaaagggattgtttctctgccaccctcataccagatctgcttcatccctgtctgaagaatgctagcccatctggctgccgatctgctatcacctgcaactctttttttatcaaggacattcccactattatgtcttctctgacctctcatcaaatcttcccattcactcaatatcccataagcatccaaactccattaaggagagttgttcaggtcactgcacaaatatatctgcaattattcatactctgtaacacttgtattaattgctgcatatgctaatacttttctaatgctgactttttaatatgttatcactgtaaaacacagaaaagtgattaatgaatgataatttagatccatttcttttgtgaatgtgctaaataaaaagtgttattaattgctggttca

SEQ ID NO: 2 - DNA, 801 bp, Artificial sequence
  TUBB rs3132584 flanking region
cccagcgcagcccaccctggcgcatcgccccccacccttgcgctggagctctctgcagccgcccgatggcctcaccccttcccgctccggtttggccctaagtaccccgcaggggggttgggcgggatgcacatgggcaaaacctcgccccgctttgtctgcgaccgtttccgcatctctctccctgcccggttctcggaccgttagaagcccttttaagtaacatcttataagtcctcggtcttcccgcccccaaacccgaagagcccttttactagtttctccaaaatgtgcctgccaagaaaaatgattcccagctttccaaaggcaaatgctagctacaattgttatatatataaatgtaacaaacttgagaggggcaaatcttgattaaggatagmgggtgcaaatgccccacaaccattttttcataacttacctggatttttccttgtaaaaagaaataaaagaggtgtaaaattcttaccttggcaccgatctggttgccacactgaccagcctggatgtgcacgatttccctcatggttaaaatttaatttttttgctcgcctcaaggtatgtatggggcaagaaaataagtaattttttttctccgcaggtcgcaggctggaaggttggaatgcgccccagaggctggagcagcgaggtgcaaacgcgacggcaggaaggttctgagagggagaaaggagaggggagggcgcggagggaaagcaggctgggcggggcgcgcgtgcgccggggctgggaggcgggagaatcggccccgcgcgcggtagggag

SEQ ID NO: 3 - DNA, 21 bp, Artificial sequence
  CYP2C8 rs1058932-specific forward primer
ataccagatctgcttcatccc

SEQ ID NO: 4 - DNA, 24 bp, Artificial sequence
  CYP2C8 rs1058932-specific reverse primer
aagatttgatgagaggtcagagaa

SEQ ID NO: 5 - DNA, 21 bp, Artificial sequence
  TUBB rs3132584-specific forward primer
caaaggcaaatgctagctaca

SEQ ID NO: 6 - DNA, 23 bp, Artificial sequence
  TUBB rs3132584-specific reverse primer
tttttacaaggaaaaatccaggt

SEQ ID NO: 7 - DNA, 45 bp, Artificial sequence
  CYP2C8-1058932-TC-SNPU
aataagctcaccaccgtcaagaagaatgctagcccatctggctgc

SEQ ID NO: 8 - DNA, 45 bp, Artificial sequence
  TUBB-3132584-CA-SNPU
gcaacataagaccgctcaacaggggcaaatcttgattaaggatag

SEQ ID NO: 9 - DNA, 18 bp, Artificial sequence
  17652121-TC-PCRU
aagagccgcctgcagaca

SEQ ID NO: 10 - DNA, 19 bp, Artificial sequence
  17652121-TC-PCRL
gctggtgcttcaggttctc

SEQ ID NO: 11 - DNA, 45 bp, Artificial sequence
  17652121-TC-SNPU
tacctatgaccagcaagcacccgtgcccatgccagacctgaagaa

SEQ ID NO: 12 - DNA, 29 bp, Artificial sequence
  754512-TA-PCRU
taaaagcaagacagttattgtttactaga

SEQ ID NO: 13 - DNA, 26 bp, Artificial sequence
  754512-TA-PCRL
ttttgtttagtgaacttttatcacca

SEQ ID NO: 14 - DNA, 45 bp, Artificial sequence
  754512-TA-SNPL
accgcactaagcaatgtatctgggatgtgactttccagaatgttt

SEQ ID NO: 15 - DNA, 20 bp, Artificial sequence
  3785883-AG-PCRU
ttgctcagcgatattgtcac

SEQ ID NO: 16 - DNA, 18 bp, Artificial sequence
  3785883-AG-PCRL
gacaccctccgggtccat

SEQ ID NO: 17 - DNA, 45 bp, Artificial sequence
  3785883-AG-SNPU
cactacatacgaccgcagaatgacaaggcactgtcaccactgggc

SEQ ID NO: 18 - DNA, 18 bp, Artificial sequence
  767058-AG-PCRU
tgaggcaaggaggggctg

SEQ ID NO: 19 - DNA, 22 bp, Artificial sequence
  767058-AG-PCRL
tagaagacaaacctggtcagag

SEQ ID NO: 20 - DNA, 45 bp, Artificial sequence
  767058-AG-SNPU
ccataacaacttaccagccagcttcctgagctctccgagctgcta

SEQ ID NO: 21 - DNA, 22 bp, Artificial sequence
  8233-AG-PCRU
ttcttaatccccaccttttctt

SEQ ID NO: 22 - DNA, 23 bp, Artificial sequence
  8233-AG-PCRL
aaaaggaagaaatgagatgttgc

SEQ ID NO: 23 - DNA, 45 bp, Artificial sequence
  8233-AG-SNPU
agaccgacaagcaatctacaaagaatgaacacccctgactctgga

SEQ ID NO: 24 - DNA, 24 bp, Artificial sequence
  242557-AG-PCRU
tagagtgtacgtttcttcttcctt

SEQ ID NO: 25 - DNA, 18 bp, Artificial sequence
  242557-AG-PCRL
tggtcccgtgacaccctg

SEQ ID NO: 26 - DNA, 45 bp, Artificial sequence
  242557-AG-SNPL
caacaagacataacaacgcaacagagccaaaaccgtgtcctggtg

SEQ ID NO: 27 - DNA, 30 bp, Artificial sequence
  2055794-AG-PCRU
tatagaacacaattccttcagagtataatc

SEQ ID NO: 28 - DNA, 25 bp, Artificial sequence
  2055794-AG-PCRL
tattgtgtgtagcatacgtccttta

SEQ ID NO: 29 - DNA, 45 bp, Artificial sequence
  2055794-AG-SNPU
gatccatcaacagacatcaccctttgtcattgaagtgtataattt

SEQ ID NO: 30 - DNA, 19 bp, Artificial sequence
  776746-AG-PCRU
tggcataggagatacccac

SEQ ID NO: 31 - DNA, 23 bp, Artificial sequence
  776746-AG-PCRL
ttcatatgatgaagggtaatgtg

SEQ ID NO: 32 - DNA, 45 bp, Artificial sequence
  776746-AG-SNPU
caacaatacgagccagcaagctttaaagagctcttttgtctttca

SEQ ID NO: 33 - DNA, 30 bp, Artificial sequence
  17110453-CA-PCRU
aaacactgaagtaaatgattctatgttaga

SEQ ID NO: 34 - DNA, 28 bp, Artificial sequence
  17110453-CA-PCRL
gcattacaatgtacattttttatacaca

SEQ ID NO: 35 - DNA, 45 bp, Artificial sequence
  17110453-CA-SNPU
agcaagaccacctagaccagttctcagattaatgaccagttggga

SEQ ID NO: 36 - DNA, 27 bp, Artificial sequence
  25527-TC-PCRU
aaaagttaggagatgattgttgtattg

SEQ ID NO: 37 - DNA, 27 bp, Artificial sequence
  25527-TC-PCRL
tatcttctttctccttcactgtgatat

SEQ ID NO: 38 - DNA, 45 bp, Artificial sequence
  25527-TC-SNPU
ccagatcctcaccatgtaagtacagaaatgtgttctgaaatctaa

SEQ ID NO: 39 - DNA, 24 bp, Artificial sequence
  15524-TC-PCRU
attgttctaaaggtggattcaaga

SEQ ID NO: 40 - DNA, 26 bp, Artificial sequence
  15524-TC-PCRL
attagattaagcccatctttatttca

SEQ ID NO: 41 - DNA, 45 bp, Artificial sequence
  15524-TC-SNPU
acaatcaacatacgaacagcagtggagaatgagttattctaagga

SEQ ID NO: 42 - DNA, 21 bp, Artificial sequence
  1061397-TC-PCRU
atcccatttagaaccaaccag

SEQ ID NO: 43 - DNA, 23 bp, Artificial sequence
  1061397-TC-PCRL
tacccactaccttctaccatttt

SEQ ID NO: 44 - DNA, 45 bp, Artificial sequence
  1061397-TC-SNPU
tacaagcacgcactagacatgctgaaaacacatgtagataatggc